(19) United States
(12) Patent Application Publication
(10) Pub. No.: US 2011/0060366 A1
(43) Pub. Date: Mar. 10, 2011

(54) Facet Joint Implant and Related Methods

(76) Inventors: Stephen Heim, Warrenville, IL (US); Luiz Pimenta, Sao Paolo (BR); Lukas Eisermann, San Diego, CA (US); Alan McLeod, Somerset (GB); Christopher Reah, Taunton (GB); Peter Butcher, Nottingham (GB)

(21) Appl. No.: 12/667,222

(22) PCT Filed: Jun. 30, 2008
(86) PCT No.: PCT/US2008/068868

§ 371 Date: Nov. 23, 2010

Related U.S. Application Data
(60) Provisional application No. 60/937,872, filed on Jun. 29, 2007.
(60) Provisional application No. 60/964,627, filed on Aug. 13, 2007.
(60) Provisional application No. 60/967,487, filed on Sep. 4, 2007.

(51) Int. Cl. A61B 17/70

(52) U.S. Cl. 606/247

(57) ABSTRACT

Implants and methods aimed at safely repairing and/or reconstructing the facet joint so as to provide the required flexibility and elasticity to support continued motion after the implant has been implanted in a facet joint.

### CROSS REFERENCES TO RELATED APPLICATIONS

The present application is an international patent application claiming the benefit of priority from U.S. Provisional Application Ser. No. 60/937,872, filed on Jun. 29, 2007, U.S. Provisional Application Ser. No. 60/964,627, filed on Aug. 13, 2007, and U.S. Provisional Application Ser. No. 60/967,487, filed on Sep. 4, 2007, the entire contents of which are hereby expressly incorporated by reference into this disclosure as if set forth fully herein. The present application also incorporates by reference the following commonly owned publications in their entireties: PCT Application Serial No. PCT/US2006/021814, entitled “Improvements Relating In and To Surgical Implants,” filed on Jun. 5, 2006; PCT Application Serial No. PCT/US2008/060944, entitled “Textile-Based Surgical Implant and Related Methods, filed Apr. 18, 2008; and U.S. Pat. No. 6,093,205, entitled “Surgical Implant,” issued Jul. 25, 2000.

### BACKGROUND OF THE INVENTION

I. Field of the Invention

The present invention relates to implants and methods generally aimed at surgery and, more particularly, to implants and methods aimed at safely repairing and/or reconstructing the facet joint.

II. Discussion of the Prior Art

Zygapophyseal joints (referred to hereafter as “facet joints”) are located between facets of the interior and superior articular processes of adjacent vertebra. Facet joints provide stability in the spine and prevent excessive torsion, while permitting a small amount of flexion, extension and lateral bending. Since facet joints are in almost constant motion with the spine, erosion of the articular processes can occur, causing spinal disorders such as degenerative spondylolisthesis or spinal stenosis.

In order to decrease the mechanical stress on the intervertebral disc due to the degenerative facet joints and stop the narrowing of the foraminal space and compressing of the spinal cord and nerves, surgeons can perform decompression and fusion. However, patients treated with decompression alone may have a risk of progressive degenerative process which can lead to further vertebral slip and/or eventual mechanical lower back pain. Although spinal fusion may reestablish stability after decompression, fusion eliminates motion altogether.

The present invention is directed at overcoming, or at least improving upon, the disadvantages of the prior art.

### SUMMARY OF THE INVENTION

The present invention accomplishes this goal by providing a motion preserving implant that, in some instances, allows for tissue and/or bony ingrowth. An implant according to the present invention is suitable for use in a variety of surgical applications, including but not limited to spine surgery. When applied to spinal surgery and implanted into a facet joint, the implant repairs/reconstructs the degenerative joint and restores the foraminal space, while advantageously preserving the natural motion of the spine. The compliant nature of the implant provides the required flexibility and elasticity to support the full range of physiological movements, as opposed to fusion surgery. In addition, the porosity and biocompatibility of the implant may facilitate tissue and/or bony ingrowth throughout part or all of the implant (if desired), which helps to secure and encapsulate the implant in the facet joint.

The implant of the present invention may be constructed in any number of suitable fashions without departing from the scope of the present invention. The implant may include a spacer and a mechanism or method for attaching the spacer within the facet joint. According to a first embodiment of the present invention, the implant includes a spacer disposed within an encapsulating jacket having a plurality of attachment flanges. To repair/reconstruct the facet joint, the spacer is positioned between a superior articular facet of an inferior vertebra and an inferior articular facet of a superior vertebra to prevent bone-on-bone contact.

A variety of materials can be used to form the spacer and/or encapsulating jacket of the implant. The spacer is preferably formed of biocompatible material. In one preferred embodiment, the spacer is formed of a textile/fabric material throughout. The spacer may be constructed from any of a variety of fibrous materials, for example including but not limited to polyester fiber, polypropylene, polyethylene, ultra high molecular weight polyethylene (UHMWPe), poly-ether-ether-ketone (PEEK), carbon fiber, glass, glass fiber, polyaramide, metal, copolymers, polyglycolic acid, polylactic acid, biodegradable fibers, silk, cellulosic and polycaprolactone fibers. The spacer may be manufactured via any number of textile processing techniques (e.g. embroidery, weaving, three-dimensional weaving, knitting, three-dimensional knitting, injection molding, compression molding, cutting woven or knitted fabrics, etc.). In another preferred embodiment, the spacer is comprised of an elastomeric component (e.g. silicon) encapsulated in fabric. In all cases, it will be understood that the spacer reduces the risk of progressive slip and the onset of lower back pain by alleviating the mechanical stress on the facet joint. Furthermore, the spacer may be provided in any number of suitable dimensions depending upon the surgical application and patient pathology.

The jacket may be constructed from any of a variety of fibrous materials, for example including but not limited to polyester fiber, polypropylene, polyethylene, ultra high molecular weight polyethylene (UHMWPe), poly-ether-ether-ketone (PEEK), carbon fiber, glass, glass fiber, polyaramide, metal, copolymers, polyglycolic acid, polylactic acid, biodegradable fibers, silk, cellulosic and polycaprolactone fibers. The jacket may be manufactured via any number of textile processing techniques (e.g. embroidery, weaving, three-dimensional weaving, knitting, three-dimensional knitting, injection molding, compression molding, cutting woven or knitted fabrics, etc.). The jacket may encapsulate the spacer fully (i.e. disposed about all surfaces of the spacer) or partially (i.e. with one or more apertures formed in the jacket allowing direct access to the spacer). The various layers and/or components of the spacer may be attached or unattached to the encapsulating jacket. The jacket may optionally include one or more fixation elements for retaining the jacket in position after implantation, including but to limited to one or more flanges extending from or otherwise coupled to the jacket and screws or other affixation elements (e.g. nails, staples sutures, tacks, adhesives, etc.) to secure the flange to an adjacent anatomical structure (e.g. vertebral body). This may be facilitated by providing one or more apertures within the flange dimensioned to receive the screws or other fixation elements.

The materials selected to form the spacer and/or jacket may be specifically selected depending upon the target location/use within the body (e.g. spinal, general orthopedic, and/or general surgical). For example in many instances it may be preferable to select UHMWPe fibers in order to generate a specific tissue response, such as limited tissue and/or bony ingrowth. In some instances it may be desirable to modify the specific fibers used, such as providing a surface modification to change or enhance a desired tissue response.

Once the spacer is implanted between the articular facets of the facet joint, attachment flanges secure the implant in situ. The attachment flanges wrap around the adjacent vertebrae and affixation elements (e.g. screws, nails, staples, sutures, buttons, bone anchors, etc.) fasten the attachment flanges to the adjacent vertebrae. The attachment flanges may be attached to any suitable portion of the vertebrae, including but not limited to the vertebral body, spinous process, pedicle, lamina, superior and/or inferior articular facet, articular process, and/or any combination thereof. It will be appreciated that any number of attachment flanges and affixation elements may be used to secure the implant in situ without departing from the scope of the present invention. In all instances, the attachment flanges (or flange) result in the implant being secured into place within the facet joint.

Although described above as having an encapsulating jacket, the implant may be presented without an encapsulating jacket. According to a second embodiment, the implant comprises a spacer with attachment flanges that are directly connected to the spacer (instead of being connected to an encapsulating jacket). In this embodiment, the spacer may also have a centrally located attachment flange. A bore is drilled completely through the superior articular process of the inferior vertebra. The centrally located attachment flange on the spacer passes through the bore in the superior articular process of the inferior vertebra and is then secured into position on the outer surface of the articular process by a fixation element(s). The attachment flanges may then be fastened to the adjacent vertebrae by affixation elements. The attachment flanges and centrally located attachment flange may be attached to any suitable portion of the vertebrae, including but not limited to the vertebral body, spinous process, pedicle, lamina, superior and/or inferior articular facet, articular process, and/or any combination thereof.

Although described herein largely in terms of attaching the spacer to the superior articular process of the inferior vertebra, it will be understood that the spacer may be attached to the inferior articular process of the superior vertebra. In all instances, the implant is situated in the facet joint and will result in the repair/reconstruction of the degenerative joint.

Any number of attachment flanges, centrally located attachment flanges or affixation elements may be used to affix the implant in situ. According to a variation of the second embodiment of the present invention, the implant may be comprised solely of the centrally located attachment flange connected to the spacer. In another variation of the second embodiment, the implant may be comprised solely of attachment flanges connected to the spacer without a centrally located attachment flange. In all instances, the implant is secured into place within the facet joint.

According to a variation of the second embodiment of the present invention, a clamping mechanism may be used to affix the spacer to the superior articular facet of the inferior vertebra. After the centrally located attachment flange of the spacer is passed through the bore in the superior articular process of the inferior vertebra, the centrally located attachment flange is passed through a hole in the middle of the clamping mechanism. The clamping mechanism slides up the centrally located attachment flange until it is pressed firmly against the outer surface of the articular process. The bolt in the clamping mechanism is then tightened to securely hold the centrally located attachment flange into place. This, in turn, anchors the implant within the facet joint. Additionally, the attachment flanges may then be fastened to the adjacent vertebrae by affixation elements.

As previously mentioned, the number of attachment flanges may be increased or decreased without departing from the scope of the present invention. Furthermore, it will be appreciated that the clamping mechanism is not limited to the second embodiment of the present invention and may be used with any embodiment of the implant described herein without departing from the scope of the present invention.

According to a third embodiment of the present invention, the implant comprises a spacer with directly attached tie cords. A bore (or bores) is drilled completely through the superior articular process of the inferior vertebra. The spacer is inserted in the facet joint while the tie cords pass through the bore in the superior articular process and are secured on the outer surface of the articular process. The tie cords may be secured through various methods, such as by way of example only, tied through a button, sutured, anchored, screwed, crimped, or any other affixation element.

Various features may be incorporated into the spacer to support the full range of physiological movements and/or limit or prevent tissue and/or bony ingrowth, for example including but not limited to an internal metal plate, a low adhesion layer (e.g. polyethylene suture thread), and/or a densely-packed substrate layer (e.g. tightly-woven nonsoluble microfibre polyester or dense embroidery). The internal metal plate of the spacer may serve to stiffen the spacer and may also serve as a radio-opaque marker, which is advantageous when tracking the implant post-surgery. In addition, the metal plate may be placed on the joint bearing surface of the spacer to help preserve motion within the facet joint by inhibiting tissue and/or bony ingrowth (as desired) due to the metallic properties. The effect of inhibiting tissue and/or bony ingrowth on the joint bearing surface is desirable and advantageous because it facilitates the free range of motion within the facet joint between the spacer and the articular facet opposite fixation. More specifically, the spacer is not attached to both articular facets thereby leaving space between the implant and one articular facet for free movement within the facet joint.

A low adhesion layer of polyethylene suture thread (or any other type of low adhesion material) may also be added to the joint bearing surface of the spacer opposite fixation. Another feature may consist of adding a non-soluble substrate layer of microfibre woven polyester (or any other non-soluble substrate material) to the joint bearing surface of the spacer opposite fixation. All of these features, whether used alone or in combination, inhibit tissue and/or bony ingrowth on the joint bearing surface due to the low adhesion and/or non-soluble aspects of the material. This effect of inhibiting tissue and/or bony ingrowth on the joint bearing surface is desirable and advantageous because it facilitates the free range of motion within the facet joint between the spacer and the articular facet opposite fixation. More specifically, the spacer is not attached to both articular facets thereby leaving space between the implant and one articular facet for free movement within the facet joint.

In addition to having tie cords, other features may be added to the spacer to help secure the implant in situ. For example, an adhesive or fusion-promoting layer (e.g. calcium hydroxyapatite, bone morphogenic protein, demineralized bone matrix, Formagraft®, stem cell material, etc.) may be added to the spacer on the surface of fixation. This adhesive layer of calcium hydroxyapatite (or any other type of adhesive material) bonds the spacer to the articular facet of fixation by facilitating tissue and/or bony ingrowth through the surface of fixation on the spacer. This effect of tissue and/or bony ingrowth on the surface of fixation is desirable and advantageous because it secures and encapsulates the implant to the inside of the facet joint.

It will be appreciated that the spacer may incorporate one or more or all of the features described above and any combination thereof without departing from the scope of the invention. It will also be appreciated that the features described above can be applied to any of the embodiments disclosed herein.

According to a fourth embodiment of the present invention, the implant may include a spacer with a guide funnel that facilitates a toggle element with tie cords. A bore (or bores) is drilled completely through the superior articular process of the inferior vertebra. The spacer is inserted in the facet joint with the guide funnel of the spacer lining up with the bore in the superior articular process. A pusher wire then pushes the toggle element through the bore in the superior articular process and next through the guide funnel of the spacer via a guide tube.

Once passed through the bore and guide funnel, the pusher wire deploys the toggle element from the guide tube to lock the spacer into position within the facet joint. The tie cords, which are attached to the toggle element, are tensioned and secured externally on the outer surface of the superior articular process of the inferior vertebra. This may be achieved by various methods, such as by way of example only, tied through a button, sutured, anchored, screwed, crimped, or any other affixation element. As a result, the toggle element and tie cords (affixed to the outer surface of the articular facet) hold the spacer securely into place within the facet joint.

According to a fifth embodiment of the present invention, the implant may include a push-on locking cap and spacer with a serrated stem (or stems). By way of example only, the stem may be made of metal or a polymer. Both the stem and push-on locking cap have serrations to facilitate secure attachment of the implant to the facet joint. Next, a bore (or bores) is drilled completely through the superior articular process of the inferior vertebra. The stem is passed through the bore in the superior articular process of the inferior vertebra, and the connected spacer is inserted between the articular facets of the facet joint.

Once the spacer and stem are placed within the facet joint, the push-on locking cap engages the stem. Due to the serrations on the inside of the push-on locking cap and the serrations on the outside of the stem, the cap can be pushed onto the stem and locked into place on the outer surface of the superior articular process of the inferior vertebra. The manner of locking the push-on cap onto the serrated stem is similar to that used in a cable tie. This may be done with a tool, such as a metal sleeve. The stem may also be trimmed to length with the excess stem being trimmed off. In all instances, the serrated stem and push-on locking cap result in the implant being secured into place within the facet joint.

According to a sixth embodiment of the present invention, the implant may include a screw-on locking cap and a spacer with a threaded stem (or stems). The screw-on locking cap may have an attached screw sleeve. By way of example only, the stem, screw-on locking cap, and screw sleeve may be made of metal or a polymer. Next, a bore (or bores) is drilled completely through the superior articular process of the inferior vertebra. The bore may be sized to fit the screw sleeve. The stem is passed through the bore in the superior articular process of the inferior vertebra, and the connected spacer is inserted between the articular facets of the facet joint.

Once the spacer and stem are placed within the facet joint, the screw-on locking cap is screwed onto the threaded stem and fixated to the outer surface of the superior articular facet of the inferior vertebra. The stem may then be trimmed to length. In addition, the base of the cap may have barbs to help facilitate fixation to the bone on the outer surface of the articular process. The barbs may be placed circumferentially in one direction. This is advantageous because it helps ensure the barbs grip to the bone surface. It will be appreciated that the feature of the barbs are not limited to this sixth embodiment and may be included in the other embodiments described herein without departing from the scope of the present invention. In all instances, the threaded stem and push-on locking cap result in the implant being secured into place within the facet joint.

According to a seventh embodiment of the present invention, the implant may include a screw and a spacer. The spacer may include a radio opaque washer plate, screw hole and cover flap. The spacer is inserted between the articular facets of the facet joint. Once implanted, the spacer is screwed directly into position in the facet joint. The screw passes through the screw hole in the spacer and is drilled into the superior articular process of the inferior vertebra. The screw is then tightened against the radio opaque washer plate in the spacer.

Once the screw secures the spacer into place, the cover flap is then folded to encapsulate the screw head. The cover flap provides additional padding and protection on the spacer between the screw and the superior articular facet of the inferior vertebra so that there is no contact between the rigid surfaces of the screw and the bone. The cover flap may include a screw hole filler that fills in the gap from the screw head to the height of the spacer. The feature of a cover flap is not limited to this embodiment only and may be included in the other embodiments of the implant described herein without departing from the scope of the present invention.

According to an eighth embodiment of the present invention, the implant may include a screw and a spacer with a screw hole, reinforced fixation hole, and mesh cover. The spacer is inserted between the articular facets of the facet joint. Once implanted, the spacer is screwed directly into position in the facet joint. The screw passes through the mesh cover and screw hole in the spacer. The screw is drilled into the superior articular process of the inferior vertebra. The screw is then tightened against the reinforced fixation hole in the spacer and the implant is secured in the facet joint.

The reinforced fixation hole in the spacer is designed to provide reinforcement in the spacer to ensure that the screw does not tear through the spacer. The mesh cover in the spacer is designed to allow the entire screw and screw head to pass through and close over it. The mesh cover then encapsulates the screw head. Although the reinforced fixation hole and mesh cover are described in this particular embodiment, it will be appreciated that these features are not limited to this embodiment and can be applied to any other embodiment described herein without departing from the scope of the present invention.

As previously described, the spacer may be formed of a textile/fabric material. By way of example only, a base textile structure may be used to form the spacer. The base textile structure is preferably manufactured via an embroidery process well known in the art using any number of biocompatible filament materials (including but not limited to polyester thread). The base textile structure may be comprised of a plurality of hinged embroidered layer regions. The mesh cover layer, which is an outer layer region of the base textile structure, is loosely constructed to allow an entire screw and screw head to pass through it. The other layer regions have screw holes to facilitate the screw fixation of the spacer into the bone. Furthermore, the base layer contains the reinforced fixation hole, which is densely embroidered to provide reinforcement in the spacer so that the screw does not tear through the spacer.

The layer regions of the base textile structure are connected together in side-by-side relation and separated by a distance to form a plurality of hinge regions between the layer regions. Then the base textile structure is then folded to form the spacer. The layer regions are folded at the hinge regions such that the layer regions are stacked together. The folding process may be performed in any number of manners as long as the mesh cover layer is placed on one outside surface of the spacer and the base layer is placed on the other outside surface of the spacer after being stacked together. It will be appreciated that any number of layer regions may be used to create the base textile structure and form the spacer without departing from the scope of the present invention. This may be done for any number of different purposes, including but not limited to varying the thickness of the spacer.

According to a ninth embodiment of the invention the implant comprises a pin element and a spacer including an attached centrally located attachment flange. Insertion of the implant is achieved by inserting the spacer within the facet joint, passing the centrally located attachment flange through an aperture spanning the targeted articular process, and finally inserting a pin element through an aperture in the central attachment flange. The central attachment flange is disposed with multiple pin element receiving apertures. Provision of multiple apertures within the central attachment flange affords the clinician the ability to select and preserve preferential central attachment flange tension and positioning, thereby preserving optimal implant positioning. Preferential spacer positioning is achieved by pulling the central attachment flange distally from the articular process, thereby exposing successive central attachment flange apertures near the articular process surface while pulling the spacer against the targeted articular facet. Once proper implant tension and positioning has been achieved, the pin element is inserted into the aperture immediately proximate to the articular process, thereby preventing central attachment flange egress into the articular process aperture, thus sustaining spacer positioning within the facet joint.

According to a tenth embodiment of the invention, the implant comprises an anchoring element and a spacer comprising an attached fixation bracket and anchorage member. Insertion of the implant begins with insertion of the spacer within the facet joint and passing the anchorage member through an aperture in the targeted articular process. Subsequently the fixation bracket is aligned with the targeted articular process and the anchorage member is inserted through a fixation bracket aperture. Preferential spacer positioning is achieved by pulling the anchorage member distally from the articular process and spacer to establish tension along the anchorage member, thereby pulling the spacer against the targeted articular facet. Anchorage member tension and spacer positioning are finally preserved through attachment of an anchoring element to the anchorage member immediately proximate to the fixation bracket thereby preventing anchorage member egress into the articular process aperture.

According to an eleventh embodiment of the present invention, the implant includes a spacer which may or may not include an encapsulating jacket as described above. Preferably, the spacer may be of textile construction (e.g. embroidered or woven), however other materials are possible, such as for example metals, plastics, glass, etc. The spacer is secured in place using a tie cord and fixation screw. The screw includes a head and a threaded shaft. The head includes a shaped engagement element dimensioned to engage an insertion device and an aperture dimensioned to allow passage of the tie cord therethrough.

In use, the tie cords function not only to secure the facet implant within the facet joint, but also to deliver the implant to the facet joint. To accomplish this, a bore is first formed through the facet surface of the superior articular process of the inferior vertebra. The tie cord is threaded through aperture of screw, and the screw is then threadedly inserted into the bore. Once the screw has been seated within the superior articular process, the tie cords are passed approximately through the middle of implant. The implant is then advanced along the tie cords into the facet joint. Once the implant has been preferentially seated within the facet joint, the tie cords may be tied to secure the implant in place, and excess tie cord may then be severed and removed.

According to a twelfth embodiment of the present invention, the implant includes a spacer and encapsulating jacket. The jacket includes a body portion having an additional pad that includes a fusion-inducing biologic agent, such as bone morphogenic protein (BMP), stem cell based material, calcium hydroxyapatite, demineralized bone matrix, or Formagraft® offered by NuVasive. The pad including the biologic agent may be provided on either side or both sides of the body portion.

In use, the implant is inserted into the facet joint such that the pads are in contact with articular processes forming the facet joint. Providing the pad on both sides encourages fusion of the implant with the facet joint. The degree of fusion that occurs may be controlled depending on the needs of the user, as described in relation to several of the examples presented above. Fusion may be achieved at least with the encapsulating jacket such that any facet motion that occurs is within the implant.

According to one embodiment of the present invention, a spacer may provided that allows for internal movement within a facet implant such as any of the examples discussed above. The spacer may be provided with or without an encapsulating jacket. The spacer is similar to those shown and described in the above-referenced '944 PCT Application. The spacer is comprised of a plurality of textile layers coupled by a plurality of hinge regions and assembled in an accordion-like manner. Other assemblies are possible, however, for example including but not limited to a plurality of individual textile layers consecutively stacked upon one another and/or a single continuous textile sheet folded upon itself to form a plurality of stacked textile layer regions. Upon assembling the spacer will comprise a pair of “outside” textile layers separated by a number of “interior” textile layers. A supplemental stitching may provided through the various textile layers to tether the layers together and increase stability of the implant.

The textile layers may be provided in any number and configuration without departing from the scope of the present invention. For example, the interior textile layers may be untreated or in the alternative treated with an anti-fusion agent in order to prevent any tissue and/or bony ingrowth through those layers. Furthermore, the layers may be chemically treated or manufactured such that they are capable of moving relative to one another. The outside textile layers are formed from or treated with fusion-inducing materials to cause tissue and/or bony ingrowth between the bone and the specific outside textile layers. The result is a facet implant including a layered spacer that achieves a textile-bone fusion interface with the facet surface of the superior articular process of a first vertebra and a textile-bone fusion interface with the facet surface of the inferior articular process of a second vertebra. However, facet motion is retained due to the capability of the interior layers to move or slide relative to one another in response to movement of the articular processes. As such, the spacer allows for a “controlled slippage” of the interior textile layers such that at least partial motion within the facet joint may be preserved. Movement of the layers is controlled due to the hinge regions and supplemental stitching as well as an encapsulating jacket (if provided), all of which function to limit the range of motion of the textile layer regions.

Many of the facet implant examples described above encourage at least some tissue and/or bony ingrowth in order to either secure the implant in place or promote complete fusion of the facet joint. Upon successful tissue and/or bony ingrowth, biodegradation, bioresorbtion, bioabsorbtion, bioabsorption, and/or bioerosion of the implant or portions thereof may be encouraged depending upon the desired motion preservation characteristics of the facet joint. For the purposes of this disclosure, bioresorbtion is meant to include any biological process (including those delineated above) in which at least a portion of the fabric component of the implant disappears or becomes detached from the rest of the implant.

According to a fourteenth embodiment of the present invention, the implant includes a spacer and encapsulating having a body portion and a plurality of attachment flanges. The encapsulating fabric of the implant includes a portion (e.g. a strip) of bioresorbable fabric on each flange adjacent to the body portion. As such, over time the bioresorbable fabric will disappear, causing the body portion and flanges to become detached from one another. The flanges may be secured to the relevant bone portions using any suitable means of attachment, for example including but not limited to bone screws, staples, sutures, nails, buttons, anchors, and/or adhesives.

Alternatively, according to a fifteenth embodiment of the present invention, the implant as described above includes portions of the encapsulating fabric forming the flanges which are entirely bioresorbable, and after resorbtion only the spacer is left within the facet joint.

According to one embodiment of the present invention, an inserter assembly may be used to insert an implant into a facet joint. In this embodiment, the inserter assembly is designed to releasably maintain the implant in the proper orientation for insertion. The implant may be introduced into a facet joint while engaged with the inserter and thereafter released. Preferably, the inserter may include a distal engagement region and an elongated handling member. The inserter may be composed of any material suitable for inserting an implant into a facet joint, including but not limited to metal (e.g. titanium), ceramic, and/or polymer compositions. According to this particular embodiment, the distal engagement region is comprised of an insertion plate. The insertion plate is generally planar rectangular in shape, but may take the form of any geometric shape necessary to interact with the implant, including but not limited to generally oval, square, and triangular. The handling member is generally cylindrical in shape. The handling member allows a clinician to manipulate the tool during an implant insertion procedure.

In order to facilitate engagement with the inserter, the spacer of the implant may include a pocket. By way of example only, the pocket may be an extra layer of embroidered fabric attached to three of the four sides of the spacer, leaving an opening for insertion of the insertion plate. The insertion plate engages with the implant by sliding into the pocket. Although slideable engagement is described herein, any suitable means of engagement may be used to engage the insertion plate with the implant, including but not limited to a threaded engagement, snapped engagement, hooks, and/or compressive force. Once the insertion plate is fit into place within the pocket of the implant, the inserter releasably maintains the implant in the proper orientation for insertion. The implant may then be introduced into a facet joint while engaged with the inserter and thereafter released. The implant, having been deposited in the facet joint, facilitates improved spinal functionality over time by maintaining a restored foraminal space (due to the structural and load-bearing capabilities of the implant) as well as enabling a desired range of motion (e.g. physiologic motion, current motion, improved motion, reduced motion, restricted motion, zero motion and/or no restriction to motion).

According to another embodiment of the present invention, an inserter assembly may include a distal engagement region comprised of two insertion prongs. Preferably, the insertion prongs are generally cylindrical in shape, but may take the form of any geometric shape necessary to interact with the implant. In order to facilitate engagement with the insertion prongs, the spacer of the implant may have attached side pockets. By way of example only, the side pockets may be made of embroidered fabric attached to each side of the spacer with openings for insertion of the insertion prongs.

The insertion prongs engage with the implant by sliding into the side pockets. Although slideable engagement is described herein, any suitable means of engagement may be used to engage the insertion prongs with the implant, including but not limited to a threaded engagement, snapped engagement, hooks, and/or compressive force. Once the insertion prongs are fit inside the side pockets of the implant, the inserter releasably maintains the implant in the proper orientation for insertion. The implant may then be introduced into a facet joint while engaged with the inserter and thereafter released. It will be appreciated that the number of insertion prongs is set forth by way of example only and may be increased or decreased without departing from the scope of the present invention. In all instances, the implant, having been deposited in the facet joint, facilitates improved spinal functionality over time by maintaining a restored foraminal space (due to the structural and load-bearing capabilities of the implant) as well as enabling a desired range of motion.

It will be appreciated that the inserter assembly and added pockets may be used with any embodiment of the implant described herein without departing from the scope of the invention. Furthermore, the inserter of the present invention is not limited to interaction with the implant disclosed herein, but rather may be dimensioned to engage any surgical implant.

### BRIEF DESCRIPTION OF THE DRAWINGS

Many advantages of the present invention will be apparent to those skilled in the art with a reading of this specification in conjunction with the attached drawings, wherein like reference numerals are applied to like elements and wherein:

FIG. 1 is a perspective view of an example of a facet implant according to a first embodiment of the present invention;

FIG. 2 is a perspective view of a spacer forming part of the implant of FIG. 1;

FIG. 3 is a perspective view of the implant of FIG. 1 positioned within a damaged facet joint;

FIG. 4 is a perspective view of two implants of FIG. 1 positioned within adjacent facet joints in one level of the spine;

FIG. 5 is a perspective view of the implant of FIG. 1 positioned within a facet joint in the spine, showing the attachment flanges secured to the adjacent vertebrae with screws;

FIG. 6 is a side view of an example of an alternative bone anchor that can be used to secure the attachment flanges of the implant of FIG. 1 to adjacent vertebrae;

FIG. 7 is a perspective view of the implant of FIG. 1 positioned within a facet joint in the spine, showing the use of the bone anchors of FIG. 6 to secure the attachment flanges to the adjacent vertebrae;

FIG. 8 is a perspective view of the implant of FIG. 1 positioned within a facet joint in the spine, showing the attachment flanges secured to the adjacent vertebrae with the bone anchors of FIG. 6;

FIG. 9 is a perspective view of the implant of FIG. 1 having two attachment flanges positioned within a facet joint in the spine and secured with screws;

FIG. 10 is a perspective view of an example of a facet implant according to a second embodiment of the present invention, having a centrally located attachment flange and without an encapsulating jacket;

FIG. 11 is a perspective view of the implant of FIG. 10 positioned within a facet joint in the spine, showing the attachment flanges secured to the adjacent vertebrae with bone anchors of FIG. 6 and the centrally located attachment flange secured with a screw;

FIG. 12 is a perspective view of the implant of FIG. 10 having a single centrally located attachment flange, according to an alternate embodiment of the implant of FIG. 10;

FIG. 13 is a perspective view of the implant of FIG. 12 positioned within a facet joint in the spine, showing the single centrally located attachment flange secured with a screw;

FIG. 14 is a perspective view of the implant of FIG. 10 in use with a clamping mechanism, according to another embodiment of the implant of FIG. 10;

FIG. 15 is a perspective view of the implant of FIG. 14 positioned within a facet joint in the spine, showing two attachment flanges secured to the adjacent vertebra with bone anchors and the single centrally located attachment flange secured with a clamping mechanism;

FIG. 16 is a perspective view of an example of a facet implant according to a third embodiment of the present invention, having tie cords in use with a button;

FIG. 17 is a side cross-sectional view of the implant of FIG. 16 positioned within a facet joint;

FIG. 18 is a perspective view of the implant of FIG. 16 positioned within a facet joint in the spine, showing the tie cords secured to the superior articular facet of the inferior vertebra with a button;

FIG. 19 is a perspective view of the implant of FIG. 16;

FIG. 20 is a side cross-sectional view of the implant of FIG. 19, showing various features of an internal metal plate, a low adhesion layer, a non-soluble substrate layer, and an adhesive layer that are part of the spacer;

FIG. 21 is a perspective view of an example of a facet implant according to fourth embodiment of the present invention, having a toggle element;

FIG. 22 is a side cross-sectional view of the implant of FIG. 21 positioned within a facet joint, showing the deployed toggle element;

FIG. 23 is a perspective view of the implant of FIG. 21 positioned within a facet joint in the spine, showing the deployed toggle element used to secure the implant to the superior articular facet of the inferior vertebra;

FIG. 24 is a perspective view of an example of a facet implant according to a fifth embodiment of the present invention, having a serrated stem and a push-on locking cap;

FIG. 25 is a side cross-sectional view of the implant of FIG. 24 positioned within a facet joint, showing the push-on locking cap secured on the stem of the spacer to the outside of the articular facet;

FIG. 26 is a perspective view of the implant of FIG. 24 positioned within a facet joint in the spine, showing the push-on locking cap and stem securing the implant to the superior articular facet of the inferior vertebra;

FIG. 27 is a perspective view of an example of a facet implant according to a sixth embodiment of the present invention, having a threaded stem and a screw-on locking cap;

FIG. 28 is a side cross-sectional view of the implant of FIG. 27 positioned within a facet joint;

FIG. 29 is a perspective view of the implant of FIG. 27 positioned within a facet joint in the spine, showing the screw-on locking cap and stem securing the implant to the superior articular facet of the inferior vertebra;

FIG. 30 is a side view of the screw-on locking cap from the implant of FIG. 27 having the added feature of barbs on the base of the cap;

FIG. 31 is a bottom plan view of the screw-on locking cap of FIG. 30, showing the barbs placed circumferentially in one direction;

FIG. 32 is a side view of a single barb on the screw-on locking cap of FIG. 31;

FIG. 33 is a perspective view of an example of a facet implant according to a seventh embodiment of the present invention, including a screw and a spacer with a cover flap;

FIG. 34 is a side cross-sectional view of the implant of FIG. 33 positioned within a facet joint;

FIG. 35 is a perspective view of the implant of FIG. 33 positioned within a facet joint in the spine, showing the screw directly securing the spacer to the inferior articular facet of the superior vertebra;

FIG. 36 is a perspective view of an example of a facet implant according to an eighth embodiment of the present invention, including a screw and a spacer with a mesh cover;

FIG. 37 is a perspective view of the implant of FIG. 36 illustrating how the screw passes through the mesh cover of the spacer;

FIG. 38 is a side cross-sectional view of the implant of FIG. 36 positioned within a facet joint, showing the screw directly securing the spacer to the inside of the articular facet;

FIG. 39 is a top plan view of a base textile structure used to form a spacer having five layer regions, one outer layer region being a mesh cover and the other outer layer region containing a reinforced fixation hole;

FIG. 40 is a top view of an inserter assembly and an implant with a pocket to facilitate engagement with the inserter assembly, according to one embodiment of the present invention for insertion of an implant into a facet joint;

FIG. 41 is top view of the inserter assembly and implant of FIG. 40 in an engaged relationship;

FIG. 42 is a top view of an inserter assembly having two prongs and an implant with side pockets to facilitate engagement with the inserter assembly, according to another embodiment of the present invention for insertion of an implant into a facet joint;

FIG. 43 is a top view of the inserter assembly and implant of FIG. 42 in an engaged relationship;

FIG. 44 is a perspective view of an example of a facet implant according to a ninth embodiment of the present invention;

FIGS. 45-46 are side partial cross-sectional views of the facet implant of FIG. 44, inserted within a facet joint and attached to the superior facet;

FIG. 47 is a perspective view of the facet implant of FIG. 44 in use with an alternate pin element;

FIG. 48 is a plan view of the pin element of FIG. 47;

FIG. 49 is a perspective view of the facet implant of FIG. 44 in use with another alternate pin element;

FIGS. 50-51 are plan views of the pin element of FIG. 49, in unassembled and assembled states, respectively;

FIG. 52 is a perspective view of an example of a facet implant according to a tenth embodiment of the present invention;

FIG. 53 is a side cross-sectional view of the facet implant of FIG. 52 inserted within a facet joint and attached to the superior facet;

FIG. 54 is a perspective view of the facet implant of FIG. 52 inserted within a facet joint of a spine;

FIGS. 55-57 are perspective views of an example of an anchoring element used to secure the implant of FIG. 52 to the facet;

FIGS. 58-60 are perspective views of an example of an alternate anchoring element of used to secure the implant of FIG. 52 to the facet;

FIG. 61 is a perspective view of an example of a facet implant according to an eleventh embodiment of the present invention, being inserted into a facet joint;

FIGS. 62-63 are perspective views of alternative examples of anchoring elements used to secure the implant of FIG. 61 to the facet;

FIG. 64 is a plan view of the implant of FIG. 61;

FIG. 65 is a perspective view of the implant of FIG. 61 inserted within a spine;

FIG. 66 is a perspective view of an example of a facet implant according to a twelfth embodiment of the present invention;

FIG. 67 is a perspective view of the implant of FIG. 66 inserted within a facet joint;

FIG. 68 is a side view of the implant of FIG. 66 inserted within a facet joint, before fusion with the bone has occurred;

FIG. 69 is a side view of the implant of FIG. 66 inserted within a facet joint, after fusion with the bone has occurred;

FIG. 70 is a perspective view of the implant of FIG. 66 inserted within a spine after fusion has occurred;

FIG. 71 is a perspective view of an example of an unfolded spacer forming part of a facet implant according to a thirteenth embodiment of the present invention;

FIG. 72 is a side view of the spacer of FIG. 71 in a folded state;

FIG. 72 is a side view of the spacer of FIG. 71 including additional stitching through the various layers to secure the spacer together;

FIGS. 74-75 are side and sectional views, respectively, of a facet implant including the spacer of FIG. 71 implanted within a facet joint, showing disposition of the various layers during flexion;

FIGS. 76-77 are side and sectional views, respectively, of a facet implant including the spacer of FIG. 71 implanted within a facet joint, showing disposition of the various layers during extension;

FIG. 78 is a perspective view of an example of a facet implant according to a fourteenth embodiment of the present invention, including flanges having biodegradable fabric portions;

FIG. 79 is a perspective view of the implant of FIG. 78 inserted within a facet joint;

FIG. 80 is a perspective view of the implant of FIG. 79 inserted with a facet joint with flanges secured to adjacent bone tissue, before degradation of the biodegradable fabric portions;

FIG. 81 is a perspective view of the implant of FIG. 80 inserted with a facet joint with flanges secured to adjacent bone tissue, after degradation of the biodegradable fabric portions;

FIG. 82 is a perspective view of an example of a facet implant including a biodegradable fabric jacket according to a fifteenth embodiment of the present invention, the facet implant inserted into a facet joint and before degradation of the biodegradable fabric jacket; and

FIG. 83 is a perspective view of the facet implant of FIG. 82 after degradation of the fabric jacket.

### DESCRIPTION OF THE PREFERRED EMBODIMENT

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The systems disclosed herein boast a variety of inventive features and components that warrant patent protection, both individually and in combination.

A variety of embodiments may be used to construct the implant of the present invention. Generally, the implant disclosed herein comprises a spacer provided with or without an encapsulating jacket. Examples of specific embodiments of the implant are described in detail below. The implant disclosed herein is suitable for use in a variety of surgical applications, including but not limited to spine surgery. When applied to spinal surgery and implanted within a facet joint, the implant repairs/reconstructs a degenerative facet joint, thereby restoring the foraminal space and preserving the natural motion of the spine. To repair/reconstruct a facet joint, the implant is positioned between a superior articular facet (of an inferior vertebra) and an inferior articular facet (of a superior vertebra) to prevent bone-on-bone contact. The compliant nature of the implant provides the required flexibility and elasticity to advantageously support the full range of physiological movements, as opposed to fusion surgery which forms a boney bridge between adjacent articular processes. In addition, the porosity and biocompatibility of the implant may facilitate tissue and/or bony ingrowth throughout part or all of the implant (if desired), which helps to secure and encapsulate the implant in a facet joint.

A variety of materials can be used to form the spacer and/or encapsulating jacket of the implant. The spacer is preferably formed of biocompatible material. In one embodiment, the spacer is formed of a textile/fabric material throughout, similar to that shown and described in commonly owned and co-pending PCT Application Serial No. PCT/US2008/060944 entitled “Textile-Based Surgical Implant and Related Methods, filed Apr. 18, 2008, the entire contents of which are hereby incorporated by reference into this disclosure as if set forth fully herein. The textile/fabric spacer may be constructed from any of a variety of natural or synthetic fibrous materials, for example including but not limited to polyester fiber, polypropylene, polyethylene, ultra high molecular weight polyethylene (UHMWPe), poly-ether-ether-ketone (PEEK), carbon fiber, glass, glass fiber, polyaramide, metal, copolymers, polyglycolic acid, polylactic acid, biodegradable fibers, nylon, silk, cellulosic and polycaprolactone fibers. The spacer may be manufactured via any number of textile processing techniques (e.g. embroidery, weaving, three-dimensional weaving, knitting, three-dimensional knitting, injection molding, compression molding, cutting woven or knitted fabrics, etc.). For the purposes of this disclosure, “textile” is meant to include any fibrous material (including but not limited to those delineated above) processed by any textile processing technique (including but not limited to those delineated above). In another embodiment, the spacer comprises at least one of an elastomer (e.g. silicon), hydrogel, hydrogel beads, plastic mesh, plastic constructs, injectable fluids, curable fluids, hair and hair constructs encapsulated in fabric, similar to that shown and described in commonly owned U.S. Pat. No. 6,093,205 entitled “Surgical Implant,” issued Jul. 25, 2000, the entire contents of which are hereby incorporated by reference into this disclosure as if set forth fully herein.

The encapsulating jacket may be constructed from any of a variety of natural or synthetic fibrous materials, for example including but not limited to polyester fiber, polypropylene, polyethylene, ultra high molecular weight polyethylene (UHMWPe), poly-ether-ether-ketone (PEEK), carbon fiber, glass, glass fiber, polyaramide, metal, copolymers, polyglycolic acid, polylactic acid, biodegradable fibers, nylon, silk, cellulosic and polycaprolactone fibers. The jacket may be manufactured via any number of textile processing techniques (e.g. embroidery, weaving, three-dimensional weaving, knitting, three-dimensional knitting, injection molding, compression molding, cutting woven or knitted fabrics, etc.). The jacket may encapsulate the spacer fully (i.e. disposed about all surfaces of the spacer) or partially (i.e. with one or more apertures formed in the jacket allowing direct access to the spacer). The various layers and/or components of the spacer may be attached or unattached to the encapsulating jacket. The jacket may optionally include one or more fixation elements for retaining the jacket in position after implantation, including but to limited to at least one flange extending from or otherwise coupled to the jacket and screws or other affixation elements (e.g. nails, staples, sutures, adhesives, tacks, etc.) to secure the flange to an adjacent anatomical structure (e.g. vertebral body). This may be facilitated by providing one or more apertures within the flange(s) dimensioned to receive the screws or other fixation elements.

The materials selected to form the spacer and/or jacket may be specifically selected depending upon the target location/use within the body (e.g. spinal, general orthopedic, and/or general surgical). For example in many instances it may be preferable to select UHMWPe fibers in order to generate a specific tissue response, such as limited tissue and/or bony ingrowth. In some instances it may be desirable to modify the specific fibers used, such as providing a surface modification to change or enhance a desired tissue response.

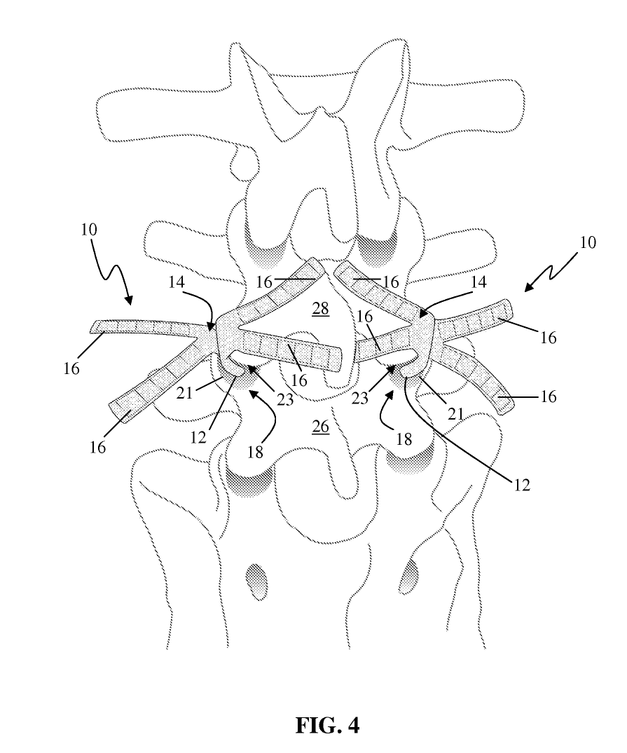

In all cases, it will be understood that the spacer disclosed herein reduces the risk of progressive slip and the onset of lower back pain by alleviating the mechanical stress on the facet joint. Furthermore, although shown in many of the examples described below as having a generally rectangular shape, the spacer may be provided in any number of suitable dimensions depending upon the surgical application and patient pathology. Furthermore, use of the implant disclosed herein is not limited to a single facet joint, but rather can be used in multiple joints at multiple levels within the spine, as needed. FIG. 4 illustrates, by way of example only, the use of two implants 10 placed in the adjacent facet joints 18 at one level of the spine.

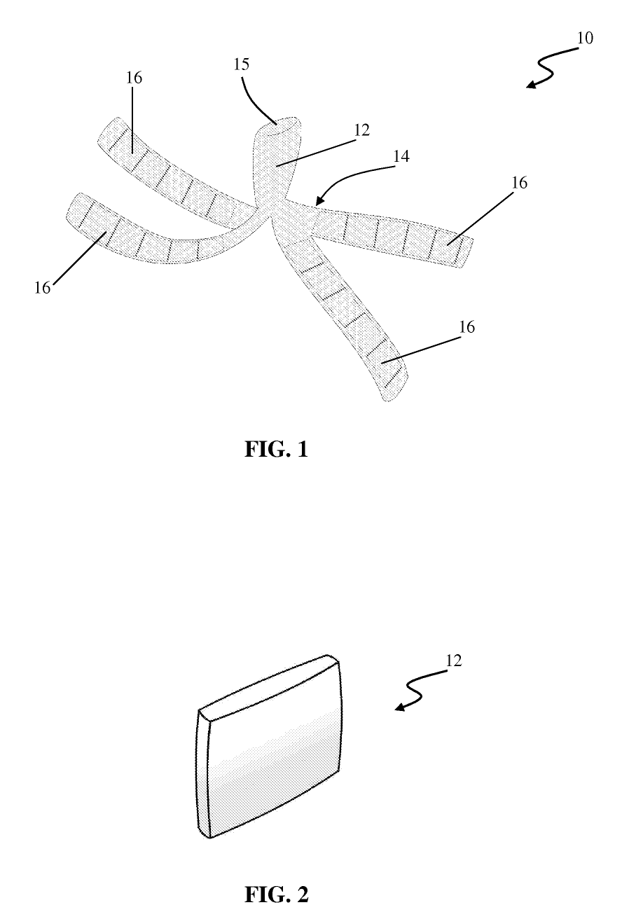

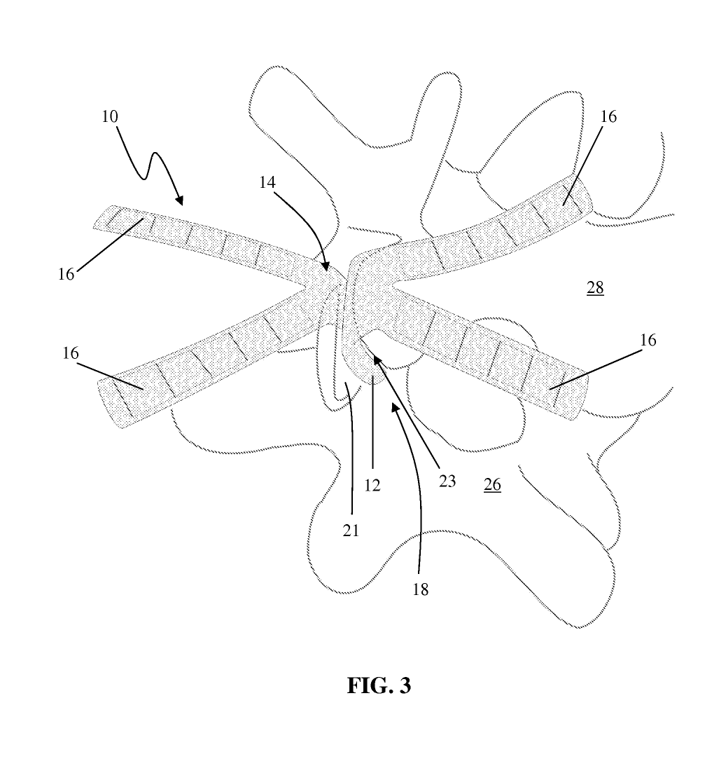

FIGS. 1-9 illustrate an example of a facet implant 10 according to a first embodiment of the present invention. Implant 10 includes a spacer 12 (shown by itself in FIG. 2) disposed within an encapsulating jacket 14 having a plurality of attachment flanges 16. In the example shown in FIG. 1, the jacket 14 includes a body portion 15 that at least partially surrounds the spacer 12. The attachment flanges 16 extend from one end of the body portion 15 such that upon insertion within a facet joint, the flanges 16 will all extend outside the joint in a similar manner. To repair/reconstruct a facet joint 18, the implant 10 is positioned between a superior articular facet 21 (of an inferior vertebra 26) and an inferior articular facet 23 (of a superior vertebra 28) to prevent bone-on-bone contact, as shown in FIG. 3.

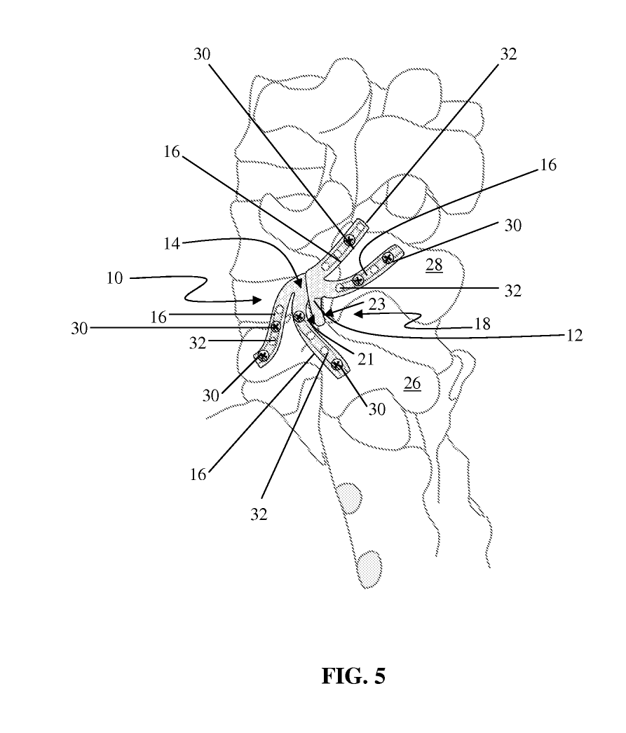

Once the spacer 12 is implanted between the articular facets 21, 23 of the facet joint 18, attachment flanges 16 secure the implant 10 in situ, as shown in FIGS. 5 & 8. The attachment flanges 16 may be constructed from any of a variety of material (e.g. polyester) via any number of techniques (e.g. embroidery). As shown in FIGS. 5 & 8 by way of example only, two attachment flanges 16 wrap around the adjacent inferior vertebra 26, and two attachment flanges 16 wrap around the adjacent superior vertebra 28. The attachment flanges 16 are then fastened to the adjacent vertebrae 26, 28 by screws 30, as shown in FIG. 5, or other affixation elements (e.g. nails, staples, sutures, buttons, anchors, etc.). The attachment flanges 16 may be attached to any suitable portion of the vertebrae, including but not limited to the vertebral body, spinous process, pedicle, lamina, superior and/or inferior articular facet, articular process, and/or any combination thereof. Any number of screws 30 or screw holes 32 in the attachment flanges 16 may be used to affix the implant 10 in situ. In a preferred embodiment, the attachment flanges 16 comprise an embroidered textile material provided with load-bearing reinforced holes 32 that are resistant to tearing under stress.

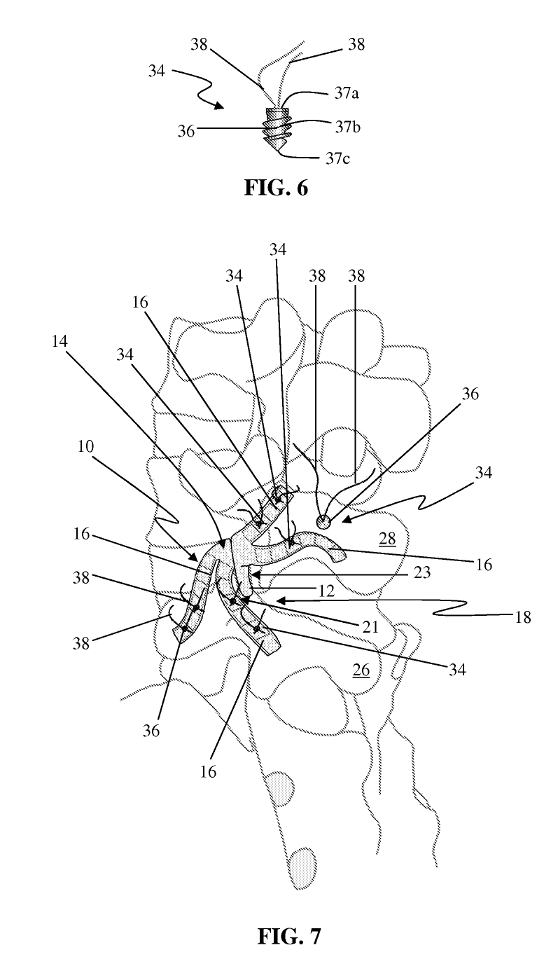

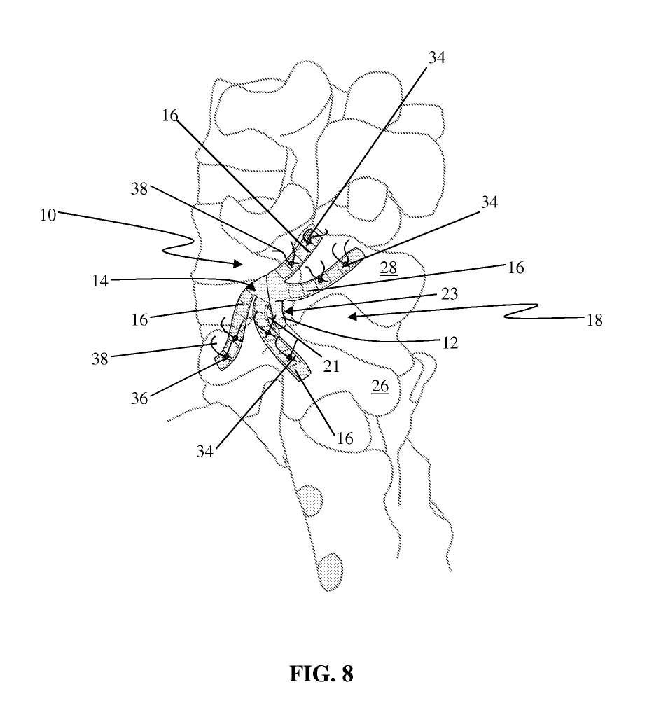

As shown in FIG. 8, alternative bone anchors 34 may be used to affix the attachment flanges 16 to the adjacent vertebrae 26, 28. FIG. 6 shows a single alternative bone anchor 34 with a metal portion 36 and sutures 38 extending therefrom. The metal portion 36 includes a proximal head region 37a, a shaft region 37b, and a distal tip 37c. The head region 37a includes an engagement element (not shown) dimensioned to engage a suitable insertion element. Examples of engagement elements include a recess, protrusion, clip, etc. The head region 37a further includes an attachment element (not shown) for facilitating attachment of the sutures 38 which extend proximally therefrom, including but not limited to (for example) a loop, clip, and/or adhesive. The shaft region 37b is preferably threaded to allow purchase within the facet bone. The distal tip 37c includes a pointed tip to allow for initial penetration into the bone. Referring to FIG. 7, the metal portion 36 of the bone anchor 34 is drilled into the vertebra 28. The sutures 38 of the bone anchor 34 slide through the attachment flanges 16 and the sutures 38 are then knotted (or tied, etc.) to secure the attachment flanges 16 to the adjacent vertebrae 26, 28.

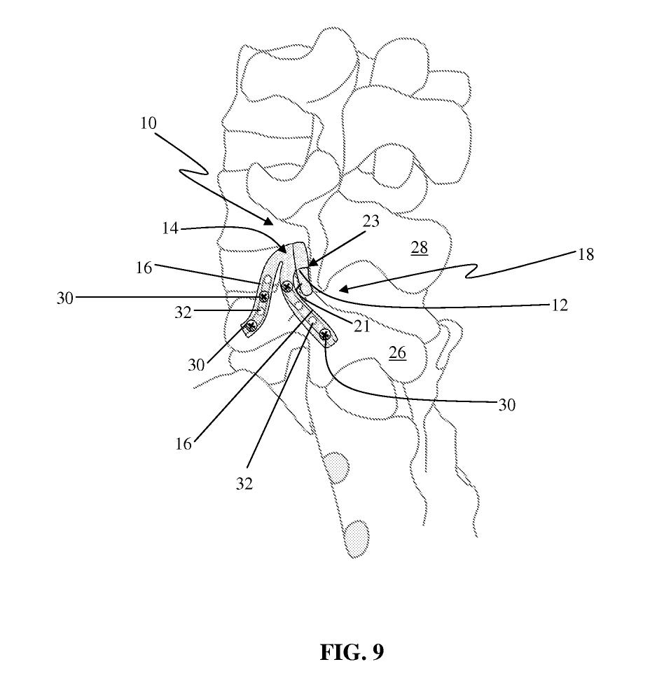

Although the implant 10 is shown in FIGS. 1-8 as having four attachment flanges 16, it will be appreciated that this is set forth by way of example only and that the number of attachment flanges may be increased or decreased without departing from the scope of the present invention. For example in FIG. 9, only two attachment flanges 16 are used to affix the implant 10 in situ. In all instances, the attachment flange(s) 16 results in the implant 10 being secured into place within the facet joint 18.

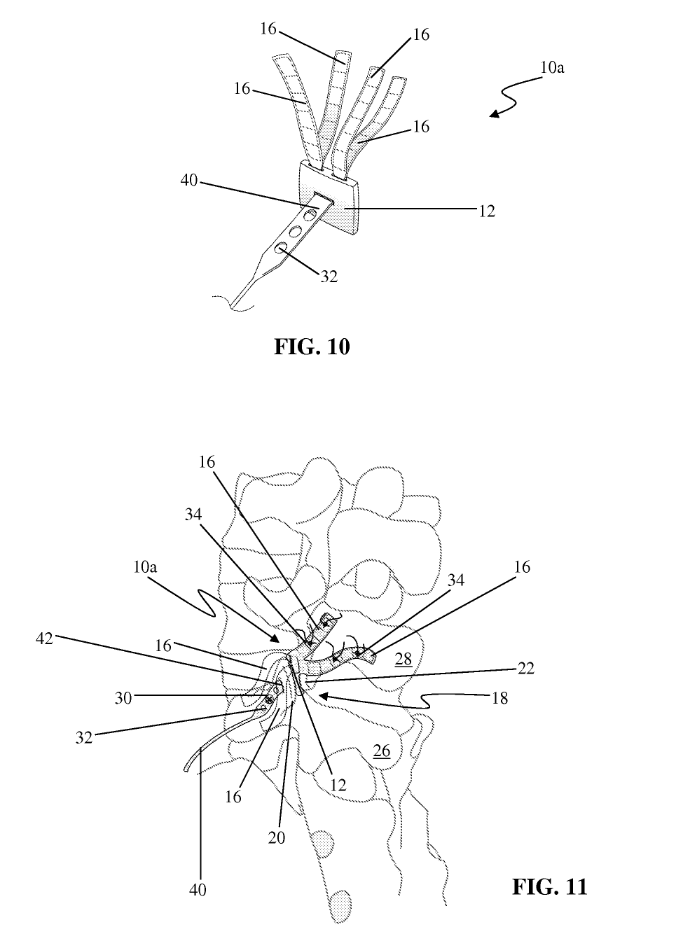

Although described above as having an encapsulating jacket 14, the facet implant of the present invention may be provided without an encapsulating jacket. For example, FIGS. 10 & 11 illustrate an example of a facet implant 10a according to a second embodiment of the present invention. The implant 10a comprises a spacer 12 with attachment flanges 16 that are directly connected to the spacer 12 (instead of being connected to an encapsulating jacket). In this embodiment, the spacer 12 may also have a centrally located attachment flange 40. A bore 42 is drilled completely through the superior articular process 20 of the inferior vertebra 26. The centrally located attachment flange 40 on the spacer 12 passes through the bore 42 in the superior articular process 20 of the inferior vertebra 26 and is then secured into position on the outer surface of the articular process by a screw 30 or any other fixation element (e.g. nails, staples, sutures, buttons, anchors, etc.). The attachment flanges 16 may then be fastened to the adjacent vertebrae 26, 28 by bone anchors 34 or other previously mentioned fixation elements. The attachment flanges 16 and centrally located attachment flange 40 may be attached to any suitable portion of the vertebrae, including but not limited to the vertebral body, spinous process, pedicle, lamina, superior and/or inferior articular facet, articular process, and/or any combination thereof.

Although described in all embodiments herein largely in terms of attaching the spacer 12 to the superior articular process 20 of the inferior vertebra 26, it will be understood that the spacer 12 may be attached to the inferior articular process 22 of the superior vertebra 28 without departing from the scope of the present invention. In all instances, the implant 10 is situated in the facet joint 18 and will result in the repair/reconstruction of the degenerative joint.

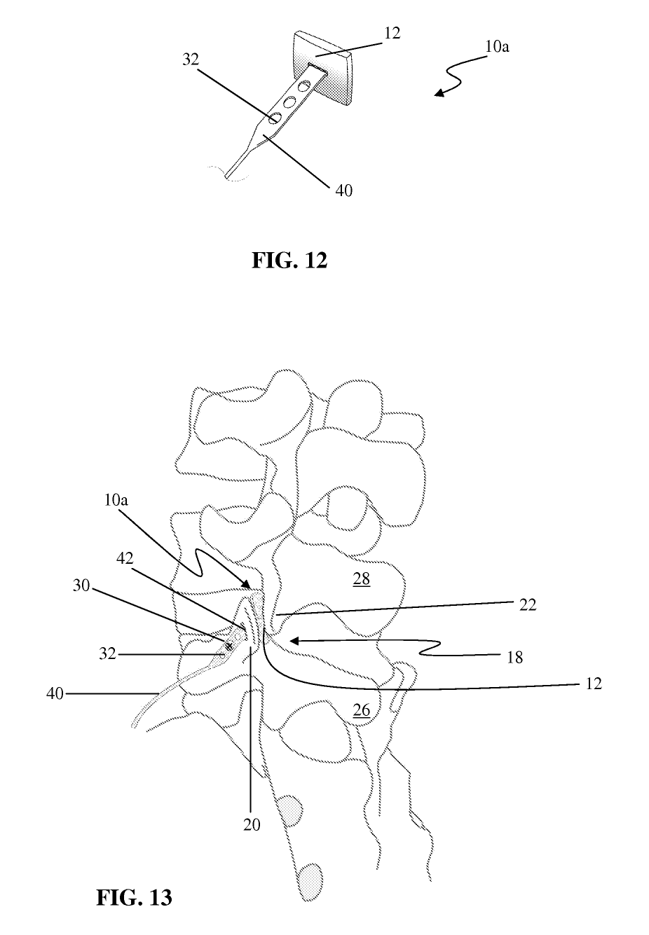

Any number of attachment flanges 16, centrally located attachment flanges 40, screw holes 32, and screws 30 or other fixation elements may be used to affix the implant 10a in situ. Although the implant 10a is shown in FIGS. 10 & 11 as having four attachment flanges 16, it will be appreciated that this number is set forth by way of example only and that the number of attachment flanges may be increased or decreased without departing from the scope of the present invention. According to a further embodiment of the present invention, as shown in FIGS. 12 & 13, the implant 10a may be comprised solely of the centrally located attachment flange 40 connected to the spacer 12. In another embodiment, the implant 10a may be comprised solely of attachment flanges 16 connected to the spacer 12 without a centrally located attachment flange 40. In all instances, the attachment flanges 16, 40 result in the implant 10a being secured into place within the facet joint 18.

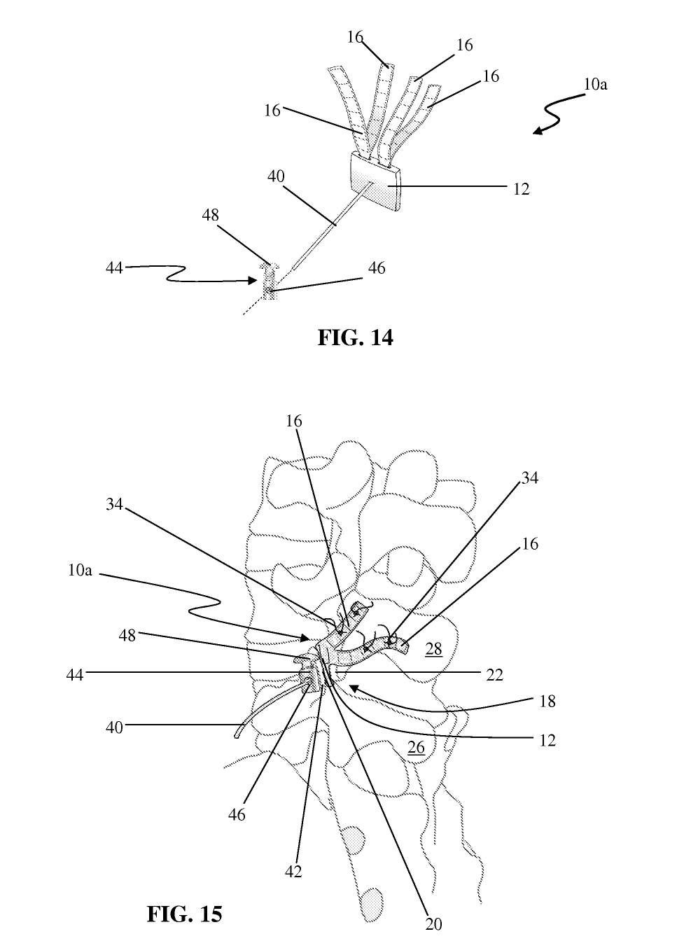

As shown in FIGS. 14 & 15 by way of example only, a clamping mechanism 44 may be used to affix the spacer 12 to the superior articular process 20 of the inferior vertebra 26. After the centrally located attachment flange 40 of the spacer 12 is passed through the bore 42 in the superior articular process 20 of the inferior vertebra 26, the centrally located attachment flange 40 is passed through the hole 46 in the middle of the clamping mechanism 44. The clamping mechanism 44 slides up the centrally located attachment flange 40 until it is pressed firmly against the outer surface of the articular process 20. The bolt 48 in the clamping mechanism 44 is then tightened to securely hold the centrally located attachment flange 40 into place. This, in turn, anchors the implant within the facet joint 18. Additionally, the attachment flanges 16 may then be fastened to the adjacent vertebrae 26, 28 by bone anchors 34 or other previously mentioned fixation elements.

Referring to FIG. 15, the implant 10a is shown having only two attachment flanges. As previously mentioned, the number of attachment flanges may be increased or decreased without departing from the scope of the present invention. Furthermore, it will be appreciated that the clamping mechanism 44 is not limited to the second embodiment of the present invention (describing implant 10a) and may be used with any embodiment of the facet implant described herein without departing from the scope of the present invention.

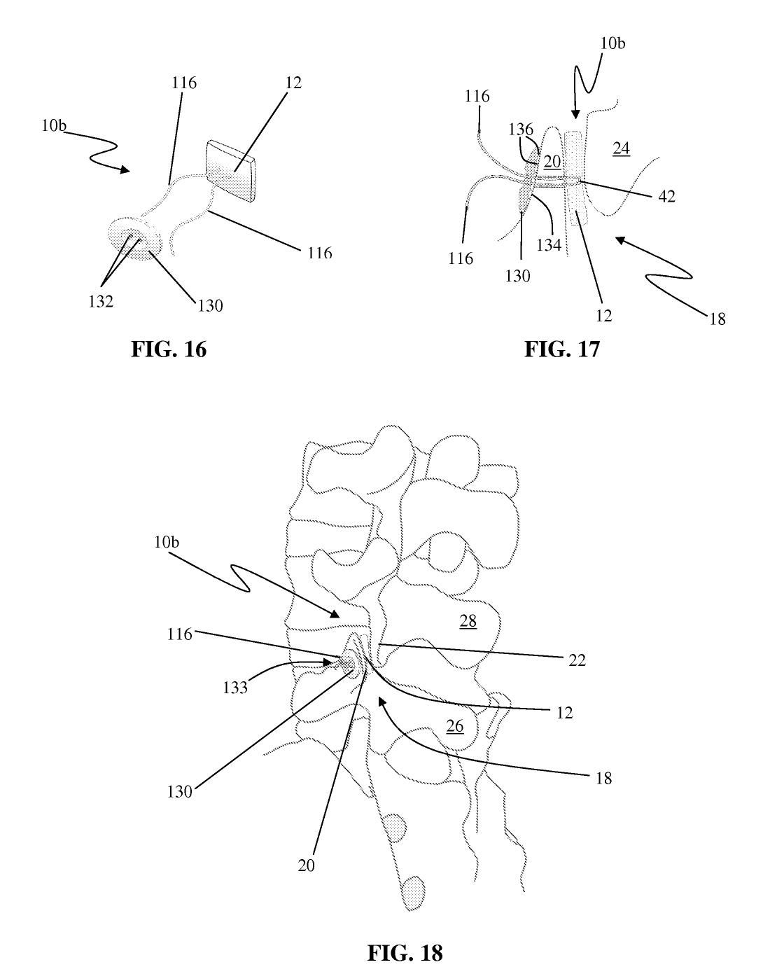

FIGS. 16-20 collectively illustrate an example of a facet implant 10b according to a third embodiment of the present invention. According to this embodiment, the implant 10b comprises a spacer 12 with directly attached tie cords 116. Tie cords 116 are preferably attached to and/or protrude from approximately the middle of one side of the spacer 12. At least one bore 42 is drilled completely through the superior articular process 20 of the inferior vertebra 26. The spacer 12 is inserted in the facet joint 18 and the tie cords 116 are manipulated to pass through the bore 42 in the superior articular process 20. The tie cords 116 are then secured on the outer surface of the articular process 20. In the example shown, the tie cords 116 are secured to the outer surface of the articular process 20 using a button 130. Button 130 includes a pair of centrally positioned apertures 132 extending therethrough, the apertures 132 dimensioned to allow passage of the tie cords 116. The tie cords 116 may then be tied together to form a knot 133 with the button positioned in between the knot and the outer surface of the articular process 20. The button further includes a bone-contacting surface 134 that are provided with anti-migration elements 136 to prevent the button from shifting relative to the bone once the knot 133 is formed. By way of example only, anti-migration features 136 may include spikes, ridges, indentations, roughness, and/or adhesives. Although shown using a button 130, the tie cords 116 may be secured through any suitable method, for example including but not limited sutures, anchors, screws, crimps, adhesives, and/or any other fixation element.

As shown in FIG. 18, the tie cords 116 are tied into a knot 133 after passage through apertures 132 in the button 130. The button 130 may be composed of any kind of material, such as metal (e.g. titanium), a polymer (e.g. a barium loaded polyester), or fabric (e.g. a densely embroidered textile plate). A metal or polymer button 130 may be roughened or spiked on its rear surface to engage with the facet bone, as shown in FIG. 17. It may also be coated with calcium hydroxyapatite to further lock to the bone. In all instances, the tie cords 116 and button 130 (or other fixation element) result in the implant 10b being secured into place within the facet joint 18.

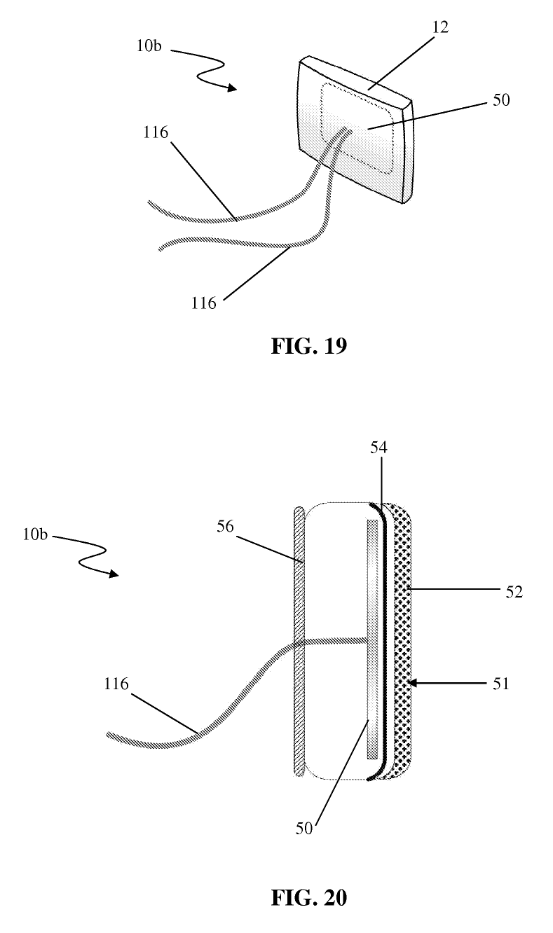

Referring to FIGS. 19 & 20, various features may be incorporated into the spacer 12 to support the full range of physiological movements and/or prevent tissue and/or bony ingrowth, for example including but not limited to an internal metal plate 50, a low adhesion layer 52 (e.g. polyethylene suture thread), a densely-packed substrate layer 54 (e.g. tightly-woven nonsoluble microfibre polyester or dense embroidery), and/or an adhesive layer 56 (e.g. calcium hydroxyapatite). The spacer 12 may contain an internal metal plate 50 which serves to stiffen the spacer and doubles as a radio-opaque marker, which is advantageous when tracking the implant 10b post-surgery. The metal plate 50 may be placed on the joint bearing surface of the spacer 12 to help preserve motion within the facet joint by inhibiting tissue and/or bony ingrowth (if desired) due to the metallic properties. The effect of inhibiting tissue and/or bony ingrowth on the joint bearing surface is desirable and advantageous because it facilitates the free range of motion within the facet joint between the spacer and the articular process opposite fixation. More specifically, the spacer is not attached to both articular processes thereby leaving space between the implant and one articular facet for free movement within the facet joint.

By way of example only in FIG. 20, a low adhesion layer 52 of polyethylene suture thread (or any other type of low adhesion material) may also be added to the joint bearing surface of the spacer opposite fixation. Another feature may consist of adding a densely-packed substrate layer 54 such as a tightly woven nonsoluble microfibre polyester (or any other densely-packed non-soluble substrate material such as a dense embroidery) to the joint bearing surface of the spacer opposite fixation. Both of these features, whether used alone or in combination, inhibit tissue and/or bony ingrowth on the joint bearing surface due to the low adhesion and/or density aspects of the material. This effect of inhibiting tissue and/or bony ingrowth on the joint bearing surface is desirable and advantageous because it facilitates the free range of motion within the facet joint between the spacer and the articular process opposite fixation. More specifically, the spacer is not attached to both articular processes thereby leaving space between the implant and one articular facet for free movement within the facet joint.

Other features affecting the degree of tissue and/or bony ingrowth are possible. For example, the surface 51 of the outer textile layer may be treated with a material that completely inhibits tissue and/or bony ingrowth such that the articulation of the implant within the joint has a textile-on-bone interface. Alternatively, any combination of the above features may be employed to encourage slight tissue and/or bony ingrowth, for example only a surface coating of tissue that is not bonded to the opposite bone, such that the articulation of the implant within the joint has a tissue-on-bone interface. Furthermore, the above features may be employed to encourage a more extensive tissue and/or bony ingrowth of tissue that is attached to the opposite articular process such that a ligament-like interface is created, with movement achieved through deformation of the tissue rather than articulation of the implant against the bone.

In addition to having tie cords 116, other features may be added to the spacer 12 to help secure the implant 10 in situ. For example as shown in FIG. 20, an adhesive layer 56 (e.g. of calcium hydroxyapatite) may be added to the spacer 12 on the surface of fixation. This adhesive layer 56 of calcium hydroxyapatite (or any other type of adhesive and/or fusion-promoting material, for example such as bone morphogenic protein, demineralized bone matrix, stem cell material, Formagraft®, etc.) bonds the spacer 12 to the facet surfaces of the articular process of fixation by facilitating tissue and/or bony ingrowth through the surface of fixation on the spacer 12. This effect of tissue and/or bony ingrowth on the surface of fixation is desirable and advantageous because it secures and encapsulates the implant 10 to the inside of the facet joint.

While FIG. 20 shows spacer 12 as including each of the features described above (i.e. metal plate 50, low adhesion layer 52, non-soluble substrate layer 54, and adhesive layer 56), it will be appreciated that the spacer 12 may incorporate one or more or all of the features, and any combination thereof without departing from the scope of the invention. Although the implant 10b shown in FIG. 20 has tie cords 116 as set forth in the third embodiment, it will be appreciated that the additional features described above can be applied to any of the embodiments described throughout this disclosure.

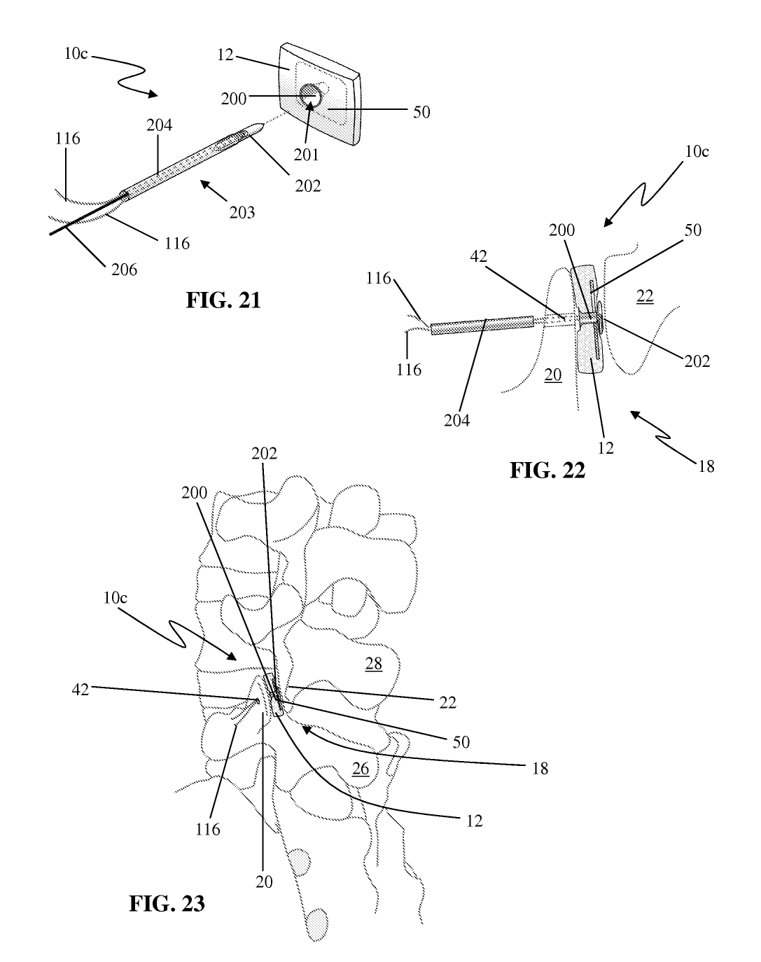

FIGS. 21-23 collectively illustrate an example of a facet implant 10c according to a fourth embodiment of the present invention. In the example shown, the spacer 12 has a generally rectangular cross-section and a fixation aperture 201 extending therethrough positioned approximately in the center thereof. The spacer 12 further includes a radio-opaque plate 50 embedded therein and a guide funnel 200 extending through aperture 201. Tie cords 116 are attached to a toggle element 202 are provided to secure the facet implant to the facet joint 18. The guide funnel 200 is configured to facilitate insertion of toggle element 202 with attached tie cords 116 through fixation aperture 201 during the securing process. The toggle element 202 is configured to toggle between an axial configuration and a normal configuration, as will be described in detail below. The radio-opaque plate 50 is included to provide intra-operative and post-operative visibility to ensure proper positioning of the facet implant 10c within the facet joint 18.

In use, at least one bore 42 is drilled completely through the superior articular process 20 of the inferior vertebra 26. The spacer 12 is inserted in the facet joint 18 with the guide funnel 200 of the spacer 12 lining up with the bore 42. An insertion device 203 consisting of a generally cylindrical elongated hollow guide tube 204 and a generally rigid pusher wire 206 is provided to facilitate insertion of the toggle element 202 and tie cords 116 through aperture 201. The toggle element 202 (with attached tie cords 116) is initially provided in an axial configuration (i.e. in axial alignment with the tie cords 116) such that the toggle element 202 may be advanced through the guide tube 204, bore 42, and ultimately aperture 201. The pusher wire 206 is provided to facilitate such advancement of the toggle element 202.

Once passed through the bore 42 and guide funnel 200, the pusher wire 204 deploys the toggle element 202 from the guide tube 204 to lock the spacer 12 into position within the facet joint 18. As the toggle element 202 emerges from the aperture 201 on the opposite side of the facet implant 10c from bore 42, the toggle element 202 will encounter the facet surface of the inferior articular process 22 of the superior vertebra 28, which will cause the toggle element 202 to toggle into a generally normal configuration (i.e. in a generally normal alignment relative to the tie cords 116). FIGS. 22 & 23 show the toggle element 202 in the deployed and locked position. Finally, the tie cords 116, which are attached to the toggle element 202, are tensioned and secured externally on the outer surface of the superior articular process 20 of the inferior vertebra 26. This may be achieved by various methods described throughout this disclosure, for example such as a button, suture, anchor, screw, crimp, or any other suitable fixation element. As a result, the toggle element 202 and tie cords 116 (affixed to the outer surface of the articular facet 20) hold the spacer 12 securely into place within the facet joint 18.

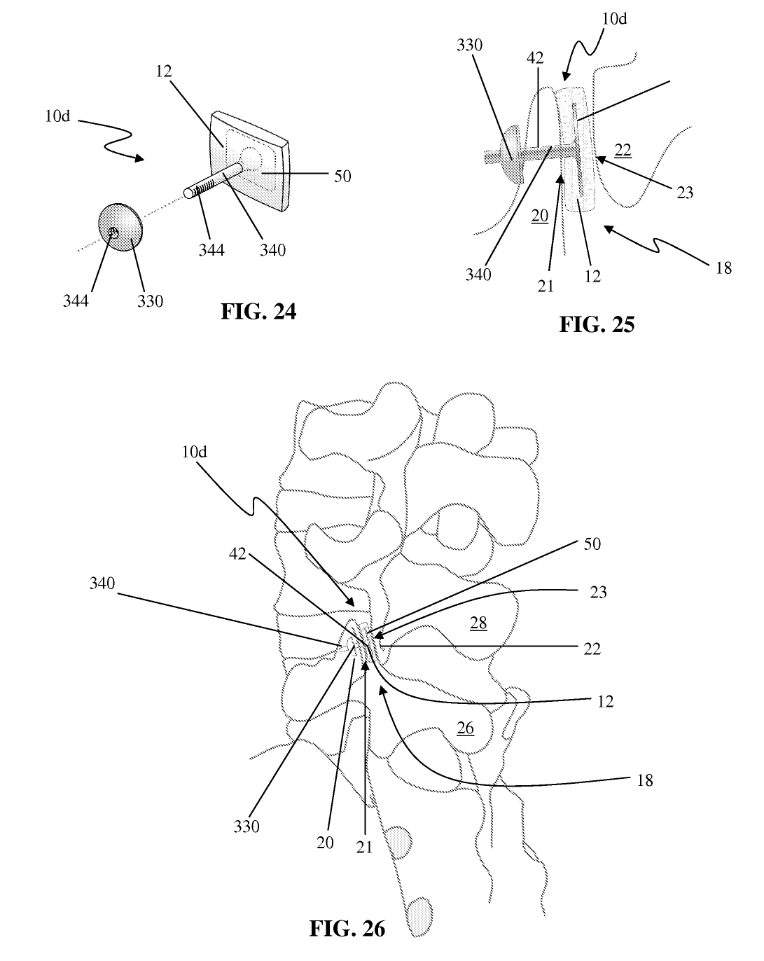

FIGS. 24-26 collectively illustrate an example of a facet implant 10d according to a fifth embodiment of the present invention. In the example shown, the implant 10d includes a spacer 12 having a generally rectangular cross-section and a radio-opaque plate 50 embedded therein. The radio-opaque plate 50 has a serrated stem (or stems) 340 extending generally orthogonally therefrom through the spacer 12. A push-on locking cap 330 is provided to engage the stem 340 and secure the implant 10d in position within the facet joint 18, as described below. By way of example only, the stem 340 may be made of metal or a polymer. Both the stem 340 and push-on locking cap 330 have serrations 344 that interact with one another to facilitate secure attachment of the implant 10d to the facet joint 18.

In use, a bore (or bores) 42 is drilled completely through the superior articular process 20 of the inferior vertebra 26. The stem 340 is passed through the bore 42 from the facet surface of the superior articular process 20 to the outside surface of the articular process 20. As a result of inserting the stem 340 through the bore 42, and because the stem 340 is attached to the radio-opaque plate 50 embedded within the spacer 12, the spacer 12 is inserted between the articular facets 21, 23 of the facet joint 18.

Once the spacer 12 and stem 340 have been inserted within the facet joint 18 as described above, the stem 340 will be protruding from the bore 42 on the outside surface of the superior articular process 20. The push-on locking cap 330 is advanced over the stem 340 to engage the outer surface of the superior articular process 20. Due to the serrations 344 on the inside of the push-on locking cap 330 and the serrations 344 on the outside of the stem 340, the cap 330 can be pushed onto the stem 340 and locked into place on the outer surface of the superior articular process 20 of the inferior vertebra 26. The manner of locking the push-on cap 330 onto the serrated stem 340 is similar to that used in a cable tie. This may be done with a tool, such as a metal sleeve. Any excess stem 340 may be trimmed to a desired length. In all instances, the serrated stem 340 and push-on locking cap 330 result in the implant 10d being secured into place within the facet joint 18.

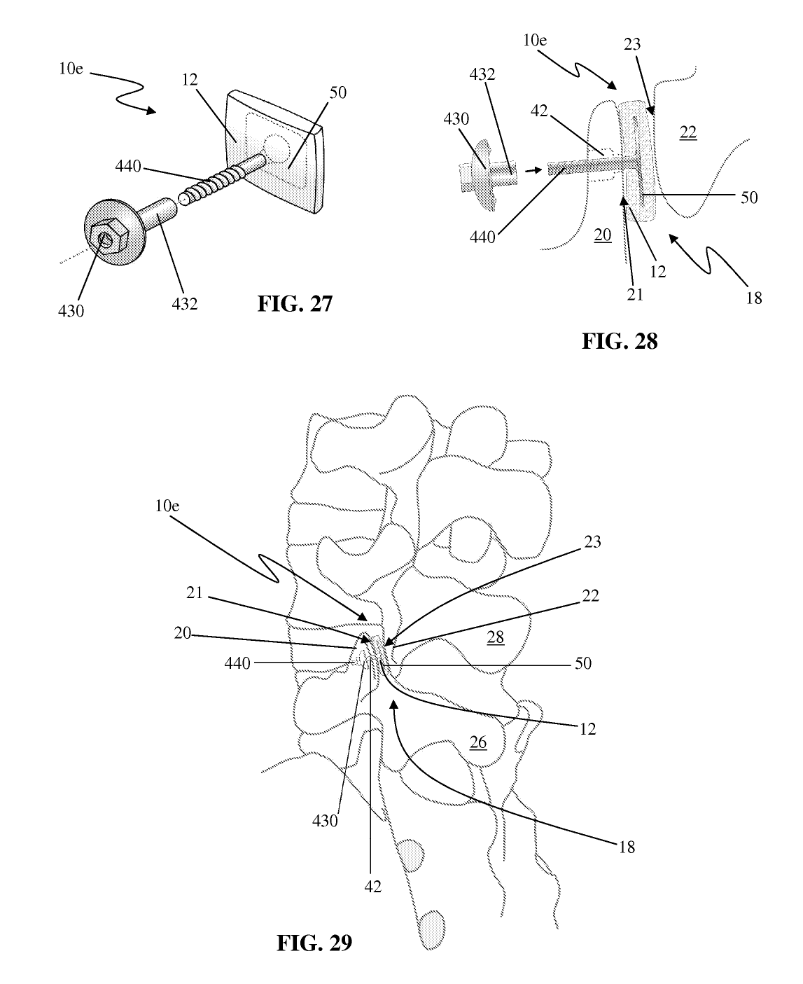

FIGS. 27-29 collectively illustrate an example of a facet implant 10e according to a sixth embodiment of the present invention. In this example, the implant 10e includes a spacer 12 having a generally rectangular cross-section and a radio-opaque plate 50 embedded therein. The radio-opaque plate 50 has a threaded stem (or stems) 440 extending generally orthogonally therefrom through the spacer 12. A screw-on locking cap 430 is provided to engage the threaded stem 440 and secure the implant 10e within the facet joint 10, as described below. The screw-on locking cap 430 has an attached screw sleeve 432. By way of example only, the stem 440, screw-on locking cap 430, and screw sleeve 432 may be made of metal or a polymer.

In use, a bore (or bores) 42 is drilled completely through the superior articular process 20 of the inferior vertebra 26. The bore 42 may be sized to fit the screw sleeve 432, as shown in FIG. 28. The stem 440 is passed through the bore 42 from the facet surface of the superior articular process 20 to the outside surface of the articular process 20. As a result of inserting the stem 440 through the bore 42, and because the stem 440 is attached to the radio-opaque plate 50 embedded within the spacer 12, the spacer 12 is inserted between the articular facets 21, 23 of the facet joint 18.

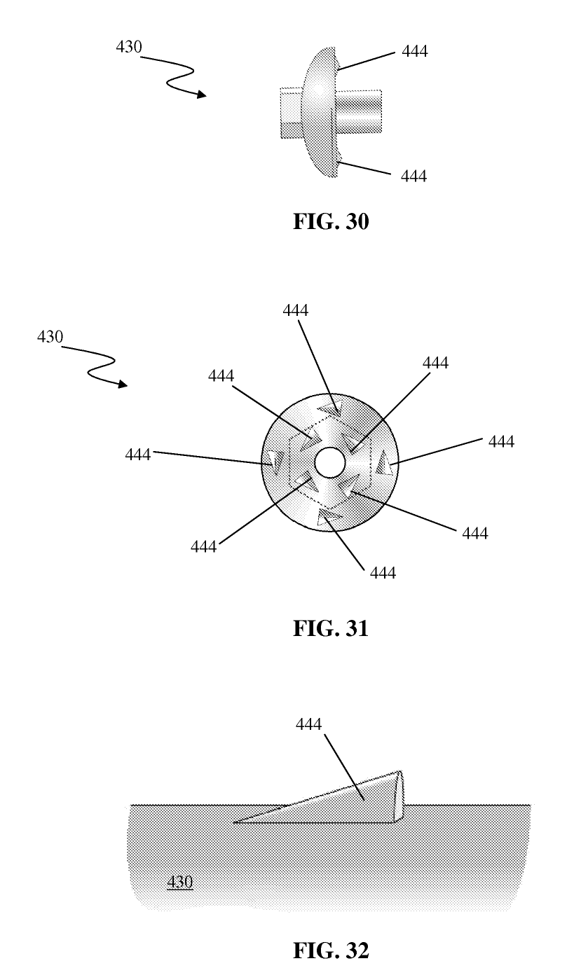

Once the spacer 12 and stem 440 have been inserted within the facet joint 18 as described above, the screw-on locking cap 430 is threadedly advanced onto the threaded stem 440 and fixed to the outer surface of the superior articular process 20 of the inferior vertebra 26. Excess stem 440 may then be trimmed to length. As shown in FIG. 30-32, the base of the cap 430 may have barbs 444 to help facilitate engagement with the bone on the outer surface of the articular process 20. The barbs 444 may be placed circumferentially in one direction, as shown in FIG. 31. This is advantageous because it helps ensure the barbs 444 grip to the bone surface. It will be appreciated that the feature of the barbs 444 are not limited to this sixth embodiment and may be included in the other embodiments described herein without departing from the scope of the present invention. In all instances, the threaded stem 440 and push-on locking cap 430 result in the implant 10 being secured into place within the facet joint 18.

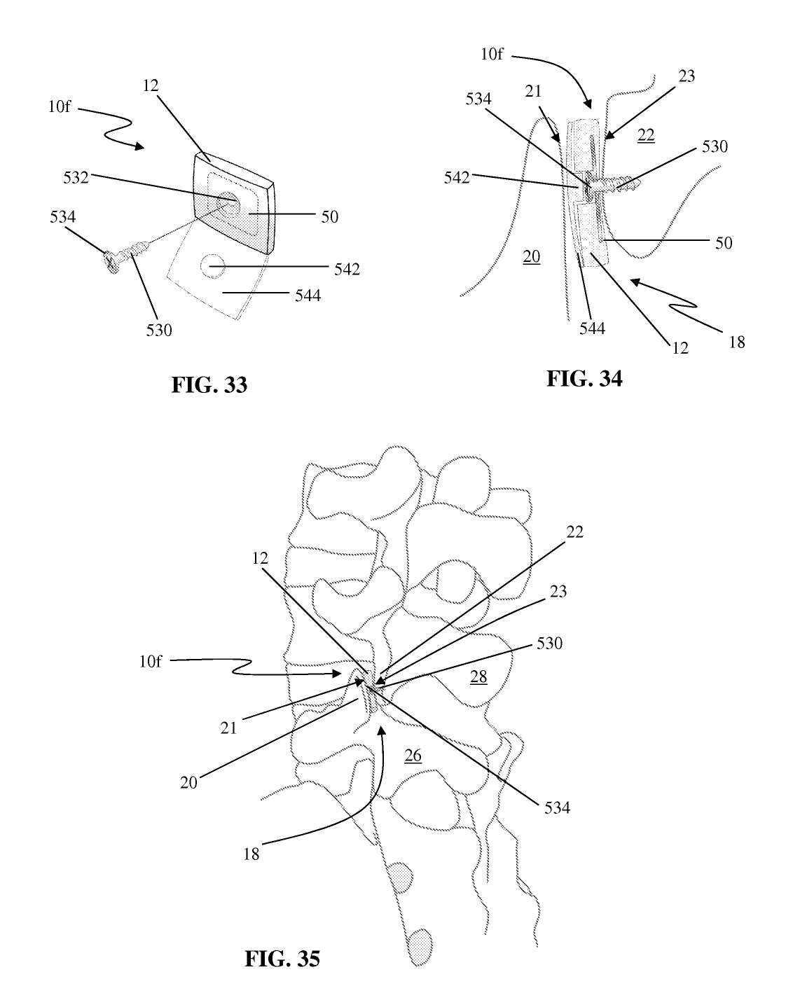

FIGS. 33-35 collectively illustrate an example of a facet implant 10f according to a seventh embodiment of the present invention. In the example shown, the implant 10f includes a spacer 12 having a generally rectangular cross-section and a screw 530 configured to attach the implant 10f to an articular process. The spacer 12 includes a radio-opaque washer plate 50 embedded therein, a screw hole 532 extending therethrough, and cover flap 544. The spacer 12 is inserted between the articular facets 21, 23 of the facet joint 18. Once implanted, the spacer 12 is screwed directly into position in the facet joint 18. The screw 530 passes through the screw hole 532 in the spacer 12 and is drilled into the inferior articular process 22 of the superior vertebra 28, as shown in FIG. 34. The screw 530 is then tightened against the radio opaque washer plate 50 in the spacer 12.

Once the screw 530 secures the spacer 12 into place, the cover flap 544 is then folded to encapsulate the screw head 534. The cover flap 544 provides additional padding and protection on the spacer 12 between the screw 530 and the inferior articular process 22 of the superior vertebra 28 so that there is no contact between the rigid surfaces of the screw and the bone. The cover flap 544 includes a screw hole filler 542 that fills in the gap from the screw head 534 to the height of the spacer 12. The feature of a cover flap 544 is not limited to this embodiment only and may be included in the other embodiments of the implant 10 described herein without departing from the scope of the present invention.

As previously described, the spacer 12 may also be attached to the superior articular process 20 of the inferior vertebra 26 without departing from the scope of the present invention. This may apply to any embodiment of the implant 10f described herein. It is understood that whether the spacer 12 is attached to the superior articular process 20 of the inferior vertebra 26 or if the spacer 12 is attached to the inferior articular process 22 of the superior vertebra 28, the implant 10f will be situated in the facet joint 18 either way and will result in the repair/reconstruction of the degenerative joint.

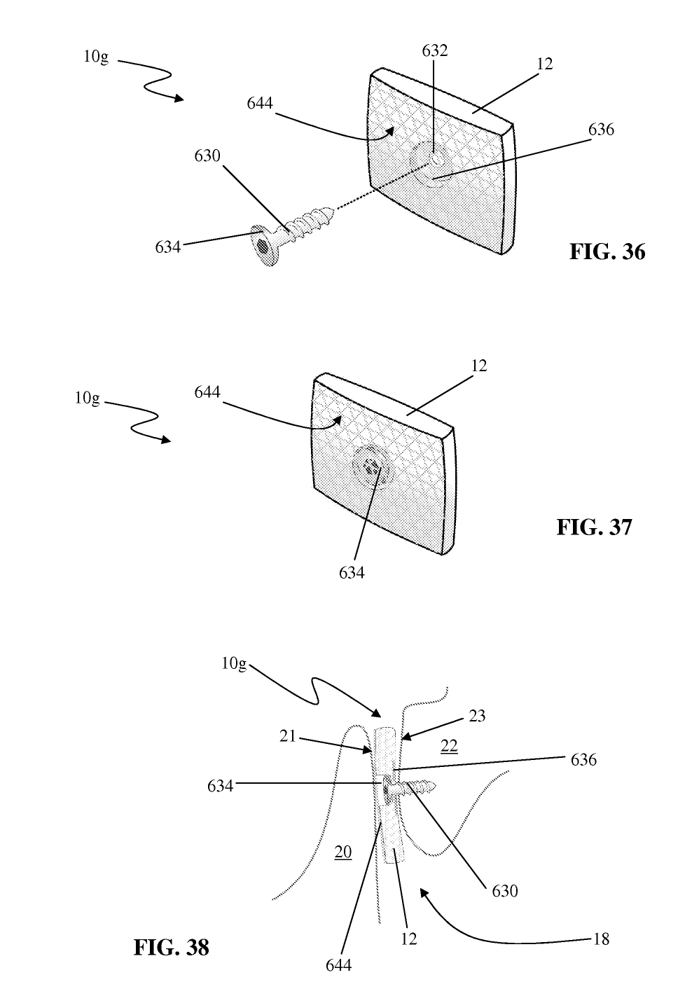

FIGS. 36-38 collectively illustrate an example of a facet implant 10g according to an eighth embodiment of the present invention. In the example shown, the implant 10g may include a screw 630 (or any other affixation element) and a spacer 12 with a screw hole 632, reinforced fixation hole 636, and mesh cover 644. The spacer 12 has a generally rectangular cross-section and is inserted between the facet surfaces 21, 23 (on articular processes 20, 22) of the facet joint 18. Once implanted, the spacer 12 is screwed directly into position in the facet joint 18. The screw 630 passes through the mesh cover 644 and screw hole 632 in the spacer 12. The screw 630 is drilled into the superior articular process 20 of the inferior vertebra 26. The screw 630 is then tightened against the reinforced fixation hole 636 in the spacer 12 and the implant 10g is secured in the facet joint 18.

The reinforced fixation hole 636 in the spacer 12 is designed to provide reinforcement in the spacer 12 to ensure that the screw does not tear through the spacer. The mesh cover 644 in the spacer 12 is designed to allow the entire screw 630 and screw head 634 to pass through and close over it. The mesh cover 644 then encapsulates the screw head 634, as shown in FIG. 37. Although the reinforced fixation hole 636 and mesh cover 644 are described in this particular embodiment, it will be appreciated that these features are not limited to this embodiment and can be applied to any other embodiment described herein without departing from the scope of the present invention.

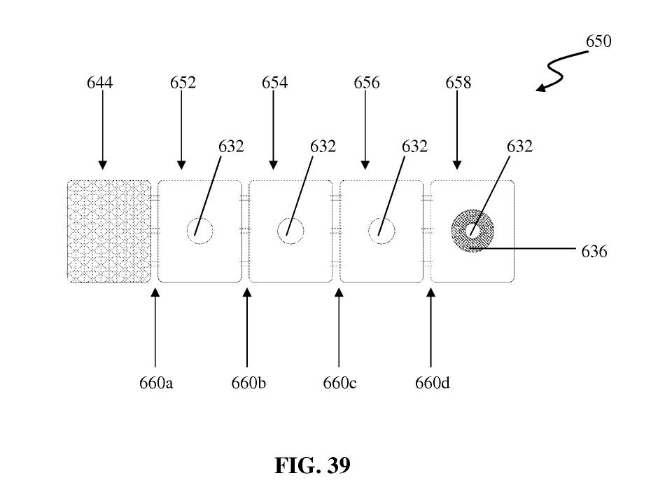

As previously described, the spacer 12 may be formed of a textile/fabric material. By way of example only, FIG. 39 illustrates a base textile structure 650 used to form the spacer 12. The base textile structure 650 is preferably manufactured via an embroidery process using any number of biocompatible filament materials (including but not limited to polyester thread). Base textile structure 650 is comprised of a plurality of hinged embroidered layer regions 644, 652-658. The mesh cover layer 644, which is an outer layer region of the base textile structure 650, is loosely constructed to allow an entire screw and screw head to pass through it. Layer regions 652-658 have screw holes 632 to facilitate the screw fixation of the spacer 12 into the bone. Furthermore, the base layer 658 contains the reinforced fixation hole 636, which is densely embroidered to provide reinforcement in the spacer 12 so that the screw does not tear through the spacer.

The layer regions 644, 652-658 of the base textile structure 650 are connected together in side-by-side relation and separated by a distance to form a plurality of hinge regions 660a-660d between the layer regions 644, 652-658, respectively. Then the base textile structure 650 is then folded to form the spacer 12. The layer regions 644, 652-658 are folded at the hinge regions 660a-660d such that layer regions 644, 652-658 are stacked together. The folding process may be performed in any number of manners as long as the mesh cover layer 644 is placed on one outside surface of the spacer 12 and the base layer 658 is placed on the other outside surface of the spacer 12 after being stacked together. It will be appreciated that the number of layer regions 644, 652-658 shown in FIG. 39 is set forth by way of example only and that the number may be increased or decreased without departing from the scope of the present invention. This may be done for any number of different purposes, including but not limited to varying the thickness of the spacer 12.

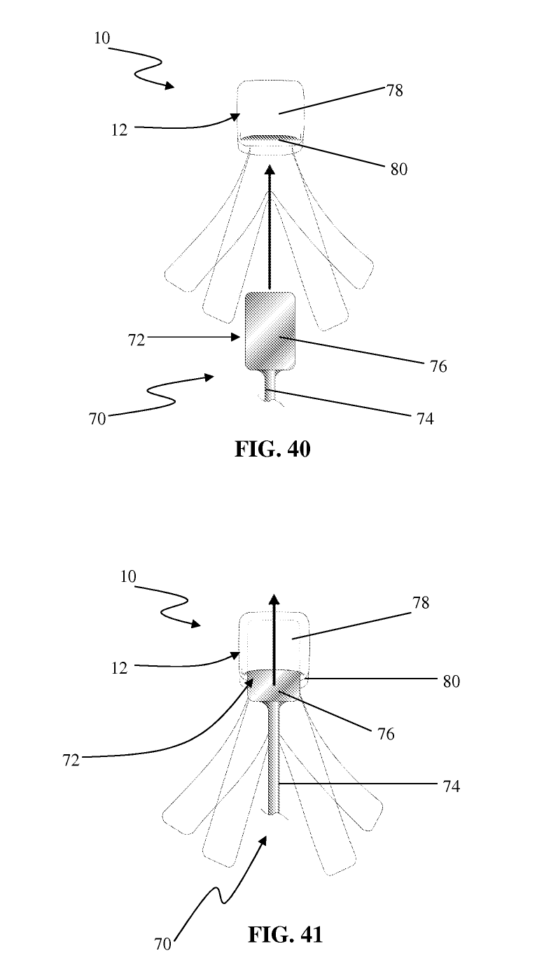

FIGS. 40 & 41 illustrate an example of an inserter assembly 70 used for inserting an implant 10 into a facet joint according to one embodiment of the present invention. The inserter assembly 70 is designed to releasably maintain the implant 10 in the proper orientation for insertion. The implant 10 may be introduced into a facet joint while engaged with the inserter 70 and thereafter released. Preferably, the inserter 70 includes a distal engagement region 72 and an elongated handling member 74. The inserter 70 may be composed of any material suitable for inserting an implant 10 into a facet joint, including but not limited to metal (e.g. titanium), ceramic, and/or polymer compositions. According to this particular embodiment, the distal engagement region 72 is comprised of an insertion plate 76. The insertion plate 76 is generally planar rectangular in shape, but may take the form of any geometric shape necessary to interact with the implant 10, including but not limited to generally oval, square, and triangular. The handling member 74 is generally cylindrical in shape. The handling member 74 allows a clinician to manipulate the tool during an implant insertion procedure.

In order to facilitate engagement with the inserter 70, the spacer 12 of the implant 10 includes a pocket 78. By way of example only, the pocket 78 is formed from an extra layer of embroidered fabric attached to three of the four sides of the spacer 12, leaving an opening 80 for insertion of the insertion plate 76. The insertion plate 76 engages with the implant 10 by sliding into the pocket 78. Although slideable engagement is described herein, any suitable means of engagement may be used to engage the insertion plate 76 with the implant 10, including but not limited to a threaded engagement, snapped engagement, hooks, and/or compressive force. Once the insertion plate 76 is fit into place within the pocket 78 of the implant 10, the inserter 70 releasably maintains the implant 10 in the proper orientation for insertion. The implant 10 may then be introduced into a facet joint while engaged with the inserter 70 and thereafter released. The implant 10, having been deposited in the facet joint 18, facilitates improved spinal functionality over time by maintaining a restored foraminal space (due to the structural and load-bearing capabilities of the implant 10) as well as enabling a desired range of motion (e.g. physiologic motion, current motion, improved motion, reduced motion, restricted motion, zero motion and/or no restriction to motion).

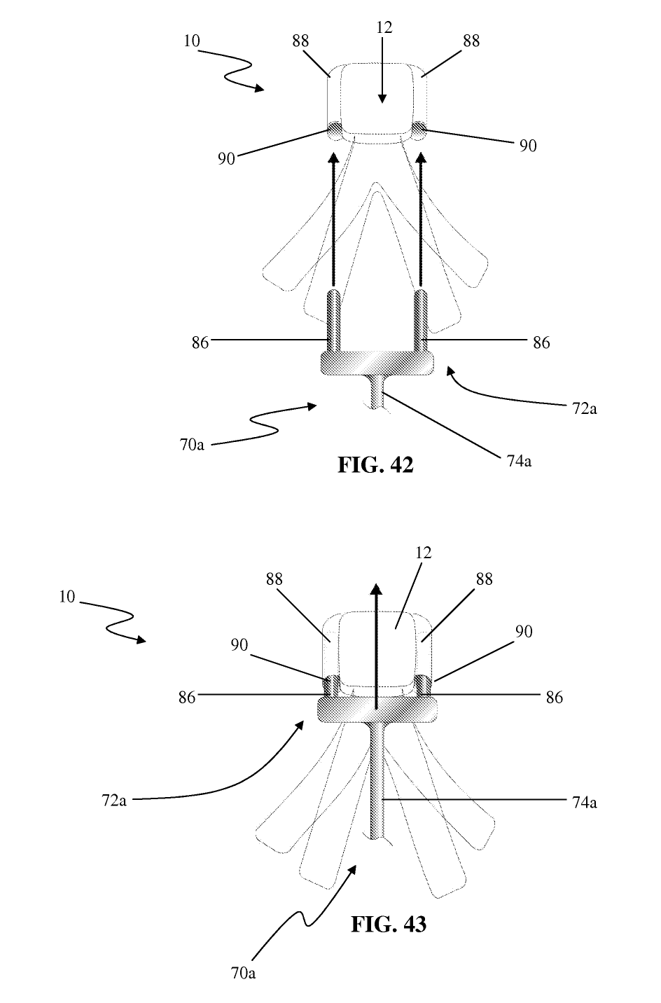

FIGS. 42 & 43 illustrate an example of an inserter assembly 70a used for inserting an implant 10 into a facet joint according to an alternate embodiment of the present invention. The inserter 70a may include a distal engagement region 72a and an elongated handling member 74a, however in this embodiment, the distal engagement region 72a is comprised of, by way of example only, two insertion prongs 86. Preferably, the insertion prongs 86 are generally cylindrical in shape, but may take the form of any geometric shape necessary to interact with the implant 10. In order to facilitate the insertion prongs 86, the spacer 12 of the implant 10 may have attached side pockets 88. By way of example only, the side pockets 88 may be made of embroidered fabric attached to each side of the spacer 12 with openings 90 for insertion of the insertion prongs 86.

The insertion prongs 86 engage with the implant 10 by sliding into the side pockets 88. Although slideable engagement is described herein, any suitable means of engagement may be used to engage the insertion prongs 86 with the implant 10, including but not limited to a threaded engagement, snapped engagement, hooks, and/or compressive force. Once the insertion prongs 86 are inside the side pockets 88 of the implant 10, the inserter 70a releasably maintains the implant 10 in the proper orientation for insertion. The implant 10 may then be introduced into a facet joint while engaged with the inserter 70a and thereafter released. It will be appreciated that the number of insertion prongs 86 is set forth by way of example only and may be increased or decreased without departing from the scope of the present invention. In all instances, the implant 10, having been deposited in the facet joint 18, facilitates improved spinal functionality over time by maintaining a restored foraminal space (due to the structural and load-bearing capabilities of the implant 10) as well as enabling a desired range of motion (e.g. physiologic motion, current motion, improved motion, reduced motion, restricted motion, zero motion and/or no restriction to motion).

It will be appreciated that although in FIGS. 40-43 the inserter assemblies 70, 70a is shown in use with the implant 10 having an encapsulating jacket and attachment flanges (as described above in the first embodiment for the implant 10), the inserter assemblies 70, 70a and added pockets 78, 88 may be used with any embodiment of the implant 10 described herein without departing from the scope of the invention. Furthermore, the inserters 70, 70a of the present invention is not limited to interaction with the implant 10 disclosed herein, but rather may be dimensioned to engage any surgical implant.

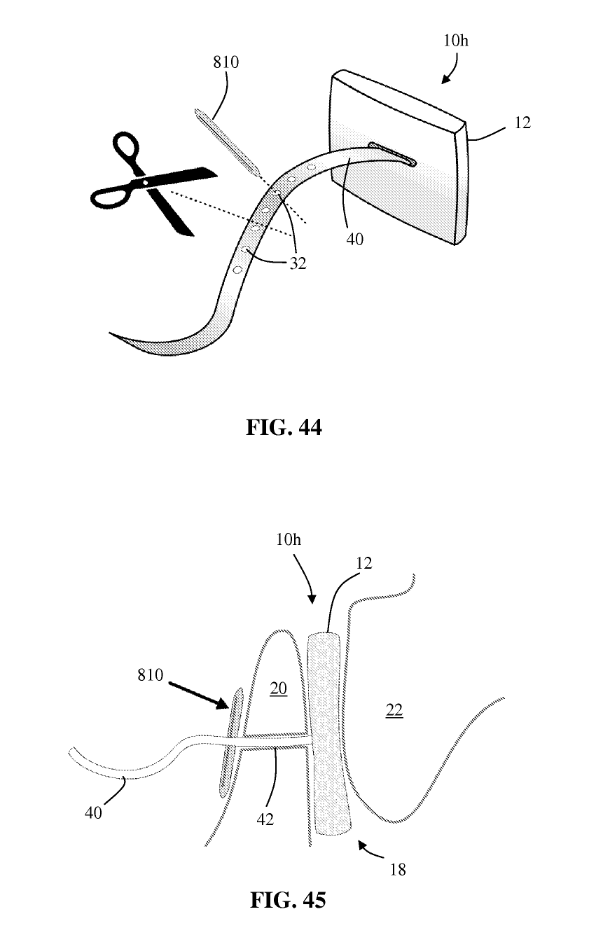

FIGS. 44-51 illustrate an example of a facet implant 10h according to a ninth embodiment of the present invention. In the example shown, implant 10h includes a spacer 12 having an attachment flange 40 extending from approximately the middle of the spacer 12, and a pin element 810 configured to secure the implant 10h in position as described below. The attachment flange 40 includes a plurality of apertures 32 through which the pin element 810 may be inserted to fix the implant in place.

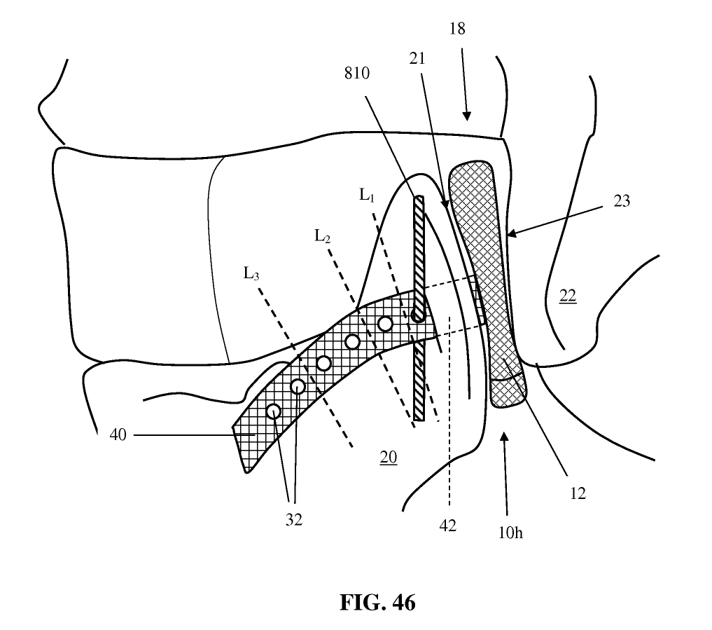

Insertion of the implant 10h is achieved through placement of the spacer 12 between the superior and inferior articular facets 21, 23 of the facet joint 18 and passing the central attachment flange 40 through a bore 42 formed through the superior articular process 20. Once inserted through the bore 42, the attachment flange 40 is pulled to apply the required tension to establish preferential seating of the spacer 12. Finally, a pin element 810 is inserted and affixed within the aperture 32 residing closest to the superior articular process 20. Properly inserted, the pin element 810 acts in conjunction with the spacer 12 to maintain a desired degree of tension on the attachment flange 40, preventing movement of the flange 40 and thereby preserving the positioning of the spacer 12 within the facet joint 18. After insertion of the pin element 810, the clinician may choose to remove any extraneous portion of the attachment flange 40 distal to the pin element 810. For example, this may be accomplished by cutting the attachment flange 40 at any number of positions including but not limited to L₁, L₂, L₃ (FIG. 46). Although presently described as inserted through the superior articular process 20 of the inferior vertebra, implantation of the implant 10h can be alternatively achieved via insertion of the attachment flange 40 through the inferior articular process 22 of the superior vertebra.

By way of example only, the attachment flange 40 extends generally orthogonally from the surface of the spacer 12. Although not shown in the attached Figures, the flange 40 may be attached to a radio-opaque plate or marker provided within the spacer 12 as described in relation to several embodiments above, and thus the flange 40 would then protrude out of the surface of the spacer 12. Alternatively, the flange 40 may be an integral extension of an encapsulating jacket provided around the spacer 12. The attachment flange 40 may be composed of any material suitable to sustain pin element 810 and spacer 12 orientations including but not limited to metal, textiles, wire, plastics, synthetic fibers and the like of any degree of flexibility. In a preferred embodiment, the attachment flange 40 comprises an embroidered textile material provided with load-bearing reinforced apertures 32 that are resistant to tearing under stress. Furthermore it can be appreciated that the attachment flange 40 may comprise any suitable dimension to afford insertion into the bore 42 while providing a sufficiently sized substrate capable of supporting an array of apertures 32 from which the clinician can choose to customize the implantation as required by the targeted insertion tissues.

The apertures 32 are distributed generally linearly along the attachment flange 40 and are dimensioned to receive the pin element 810. It can be appreciated that any number of apertures 32 may be disposed in any pattern within the attachment flange 40 which might align with preferential receiving tissue. Furthermore, the apertures 32 may be either reinforced or not reinforced dependent upon the likely compositional interactions between the pin element 810 and attachment flange 40.

The pin element 810 may comprise any configuration and composition suitable to sustain pin element 810 positioning within the aperture 32 while also sustaining proper spacer 12 positioning within the facet joint 18. Examples of suitable configurations of pin element 810 include but are not limited to crimps, textile or wire ties, male/female coupler elements, snaps, screws and the like which might be detachably or permanently inserted into the aperture 32. Furthermore it can be appreciated that the pin element 810 may be composed of any suitable material capable of preserving preferential implant 10 positioning within the facet joint 18 including but not limited to metal, plastic, textiles, synthetic fibers and the like.

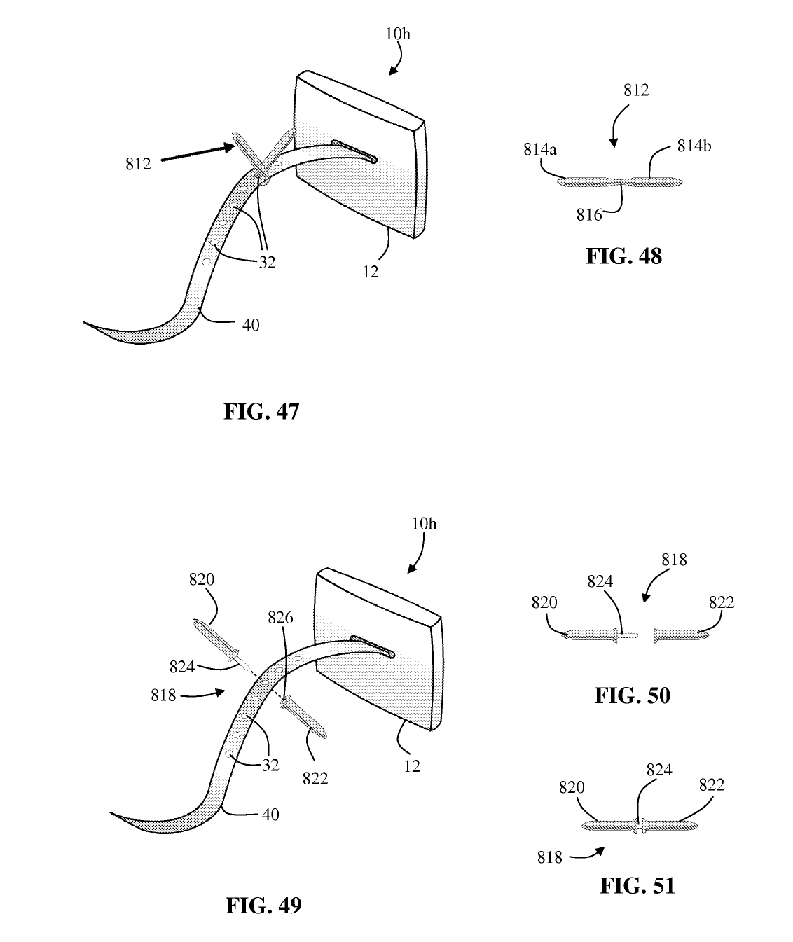

Moreover, while pin element 810 shown in FIGS. 44-46 is a single piece, generally rigid construct, other configurations of pin elements are possible. For example, FIGS. 47-48 disclose an example of a bendable pin element 812, and FIGS. 49-51 illustrate an example of a multi-piece pin element 818. Referring first to FIGS. 47-48, pin element 812 is shown in use with a facet implant 10h as described above. Pin element 812 is generally elongated and may have any cross-sectional shape, including but not limited to circular, ovoid, square, rectangular, triangular, etc. Pin element 812 includes a pair of end portions 814a, 814b separated by a bendable central portion 816. The pin element 812 is initially provided in an unbended, linear configuration as shown in FIG. 48. After spacer 12 of implant 10h has been inserted into the facet joint as described above, pin element 812 is inserted through an aperture 32 provided within attachment flange 40. When the central portion 816 is aligned with the opening of the aperture 32, the central portion 816 is bent such that the end portions 814a, 814b are no longer in a linear relationship to one another. Central portion 814 may be bent to any degree desirable. The bending of the pin element 812 helps ensure that the pin element 812 remains in place within aperture 32 and consequently that adequate tension is maintained on flange 40 to keep spacer 12 in position within the facet joint.

Referring to FIGS. 49-51, an example of an alternative pin element 818 is described. In this example, pin element 818 comprises a first pin element 820 and a second pin element 822. Pin elements 820, 822 are generally elongated, generally rigid, and may have any cross-sectional shape, including but not limited to circular, ovoid, square, rectangular, triangular, etc. First pin element 822 includes a post 824 projecting axially from one end. Second pin element 824 includes a recess 826 formed within one end, the recess 826 being of a shape complementary to that of the post 824, and further dimensioned to securely receive the post 824 in order to create a locked relationship relative to one another. Such a locked relationship may be accomplished through a threaded interaction, friction fit, and/or adhesive material. Upon mating of the first and second pin elements 820, 822, a portion of the post 824 remains exposed (FIG. 51) to account for the thickness of the attachment flange 40. In use, the pin element 818 is initially provided as separate first and second pin elements 820, 822. After spacer 12 of implant 10h has been inserted into the facet joint as described above, post 824 of first pin element 820 is inserted through an aperture 32 provided within attachment flange 40. Recess 826 of pin element 822 is then aligned with and advanced over post 824 until the first and second pin elements 820, 822 are suitably locked together. The result is a generally rigid pin element 818 functioning similarly to pin element 810 described above. One benefit to a multi-piece pin element 818 as described is that the apertures 32 need only be large enough to permit passage of post 824 therethrough, thus potentially increasing the load-bearing capacity of the flange 40, or conversely reducing the amount of material necessary for flange 40 construction.

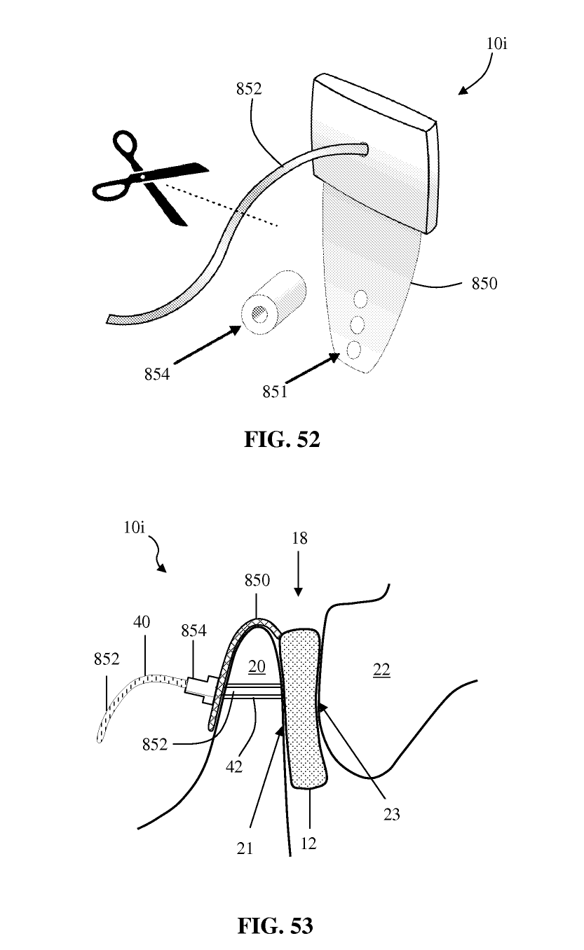

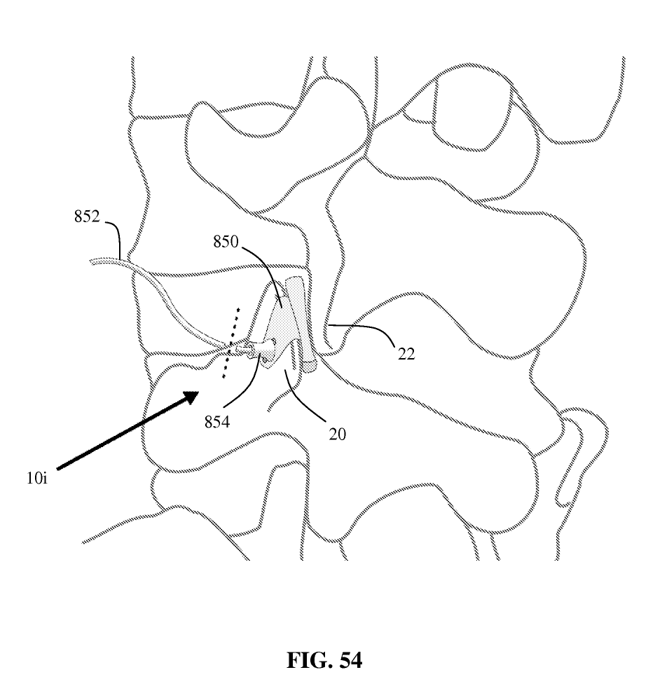

FIGS. 52-60 illustrate an example of a facet implant 10i according to a tenth embodiment of the present invention. Facet implant 10i comprises an anchoring element 854 and a spacer 12, as previously presented herein, including an attached fixation bracket 850 and anchorage member 852. The fixation bracket 850 is attached to the spacer 12 and configured to extend around an extent of the superior articular process 20 to at least fractionally engage the outer surface of the superior articular process 20. Additionally the fixation bracket 850 includes at least one aperture 851 dimensioned to receive the anchorage member 852 therethrough. Proper insertion of the implant 10i is achieved through insertion of the spacer 12 within the facet joint 18, and passing the anchorage member 852 through a bore 42 which extends through the superior articular process 20. Implantation is completed by positioning the fixation bracket 850 over an extent of the superior articular process 20 such that the relevant aperture 851 is in general alignment with bore 42, passing the anchorage member 852 through the aperture 851, applying the desired tension to the anchorage member 852, and finally affixing an anchoring element 854 to the anchorage member 852 at some point proximate to the fixation bracket 850. Preferably, the anchoring element 854 is cinched into a snug interaction with the fixation bracket 850. Subsequent to attaching the anchorage element 854, the anchorage member 852 may be trimmed at any point distal to the anchorage element 854, as indicated in FIG. 54. Although presently described as inserted through the superior articular process 20 of the inferior vertebra, it can be appreciated that implantation of the implant 10 can be alternatively achieved via insertion of the anchorage member 852 through the inferior articular process 22 of the superior vertebra.

The fixation bracket 850 is dimensioned to extend around an extent of and engage the outer surface of the superior articular process 20. The fixation bracket 850 may comprise one or more apertures 851 disposed in any number of configurations sufficient to provide a clinician the opportunity to preferentially orient the fixation bracket 850 with the inserted anchorage member 852. Therefore it can be appreciated that the fixation bracket 850 of the present invention may comprise any suitable dimension which will afford optimal engagement of the superior articular process 20 while also providing a sufficiently sized substrate capable of supporting one or more apertures 851. Moreover the fixation bracket 850 may comprise any suitable material of sufficient strength and flexibility with which to support spacer 12 and anchorage member 852 positioning including but not limited to pliable or inflexible metal, textile, plastic, synthetic materials and the like. In a preferred embodiment, the fixation bracket 850 comprises an embroidered textile material provided with load-bearing reinforced apertures 851 that are resistant to tearing under stress.

The anchorage member 852 of the present embodiment comprises a generally pliable shaft extending from the surface of the spacer 12 and dimensioned to pass through apertures 42 and 851 and the anchoring element 854. Although described as generally pliable, the anchorage member 852 may be composed of material exhibiting any degree of flexibility while being of suitable strength to hold the implant 10 in place including but not limited to pliable or inflexible metal, textile, plastic, synthetic fibers (e.g. woven or embroidered) and the like. Furthermore the anchorage member 852 may be of any suitable length which provides clinicians with the ability to customize insertion and positioning of the implant 10 as directed by the structure of the receiving tissues. Additionally the anchorage member 852 may constitute any dimension and/or surface structures including but not limited to textures and/or treatments, to provide for optimal anchoring element 854 engagement with the anchorage member 852.

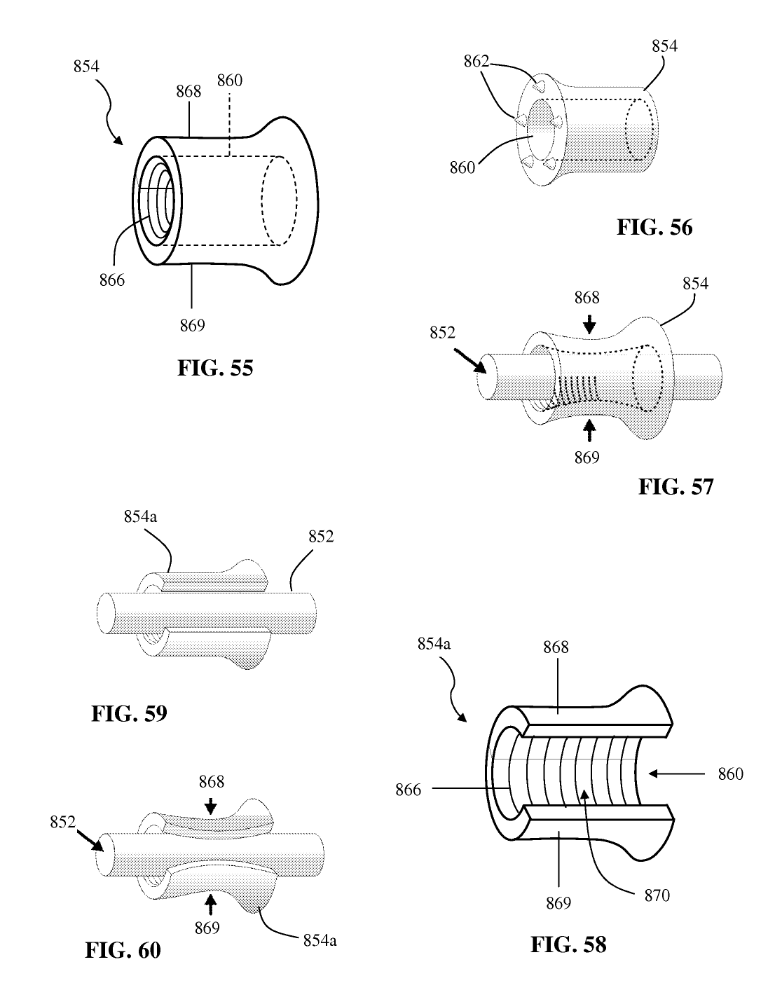

FIGS. 55-58 illustrate one example of an anchoring element 854. Anchoring element 854 includes a textured lumen 860 into which the anchorage member 852 is introduced. Lumen 860 has a cross-sectional shape generally corresponding to the shape of the anchorage member 852. Texture 866 on the interior of lumen 860 may comprise (for example) a plurality of ridges, threads, protrusions, etc. Once anchorage member 852 is introduced through lumen 860, it is secured via compression of outer anchoring element surfaces 868, 869, as shown in FIG. 57. Generally optimal implant placement is achieved by tensioning the anchorage element 852 to create preferential engagement of the spacer 12 with the superior articular facet 21 (FIG. 53), and then affixing the anchoring element 854 to the anchorage member 852 and against the surface of the fixation bracket 850, thereby securing the position of spacer 12 within the facet joint 18. Anchoring element 854 may further include a plurality of engagement features 862 on the leading end, dimensioned to engage the fixation bracket 850 to ensure minimal relative movement between anchoring element 854 and fixation bracket 850.

FIGS. 58-60 illustrate an example of an alternative anchoring element 854a. Anchoring element 854a has the same features of anchoring element 854 except that it includes a break 870 in the side to enable the anchorage member 852 to pass through and enter the lumen 860. As with anchoring element 854, anchoring element 854a includes texture 866 on the interior of lumen 860, which may comprise (for example) a plurality of ridges, threads, protrusions, etc. Once anchorage member 852 is introduced through lumen 860, it is secured via compression of outer anchoring element surfaces 868, 869, as shown in FIG. 60. Although not shown, anchoring element 854a may include a plurality of engagement features on the leading end, dimensioned to engage the fixation bracket 850 to ensure minimal relative movement between anchoring element 854 and fixation bracket 850.

Although illustrated as having a crimp-like configuration, the anchoring element 854 may comprise any number of suitable configurations including but not limited to detachably or permanently applied screws, ratcheting rivet assemblies and other suitable devices for engaging the anchorage member 852 while restricting anchoring member 854 movement. Furthermore, the anchoring element 854 may be composed of any suitable material capable of engaging and sustaining anchorage member 852 positioning therein including but not limited to metal, textile, plastic, synthetic fibers and the like.

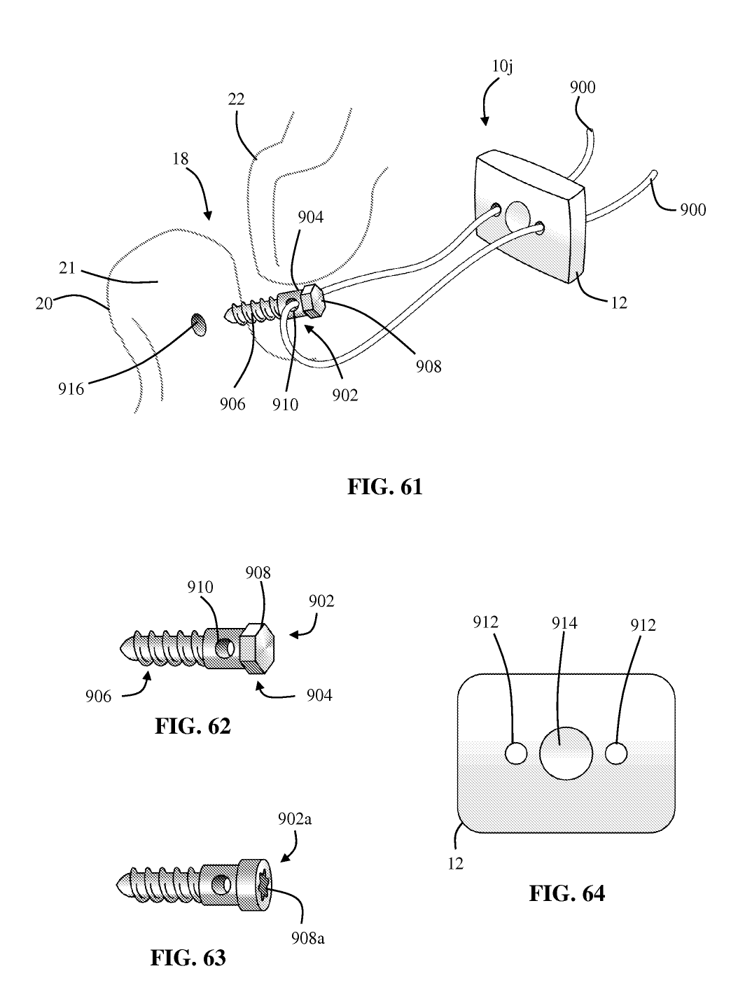

FIGS. 61-65 illustrate an example of a facet implant 10j according to an eleventh embodiment of the present invention. Facet implant 10j includes a spacer 12 which may or may not include an encapsulating jacket as described above. Preferably, spacer 12 may be of textile construction (e.g. embroidered or woven), however other materials such as those described above are possible. Facet implant 10j is has a generally rectangular cross-section and is dimensioned to be inserted within a facet joint 18 between a superior articular process 20 of a first vertebra and an inferior articular process 22 of a second vertebra. Spacer 12 is secured in place using a tie cord 900 and fixation screw 902. As illustrated in FIG. 62, screw 902 includes head 904 and a threaded shaft 906. Head 904 includes a shaped engagement element 908 dimensioned to engage an insertion device (not shown) and an aperture 910 dimensioned to allow passage of the tie cord 900 therethrough. An alternative example of a screw 902a is provided in FIG. 63. Screw 902a is similar to screw 902, except that the head 904 includes a shaped recess 908a dimensioned to receive an insertion device (not shown), such as a screw driver.

As illustrated by way of example only in FIG. 64, spacer 12 is generally rectangular in shape and has a pair of apertures 912 and a recess 914. Apertures 912 extend completely through the spacer 12 and are dimensioned to receive the tie cords 900 therethrough. The recess 914 is positioned in the middle of the spacer 12 and is dimensioned to at least partially receive the head 904 of the screw 902 upon implantation in the facet joint 18.

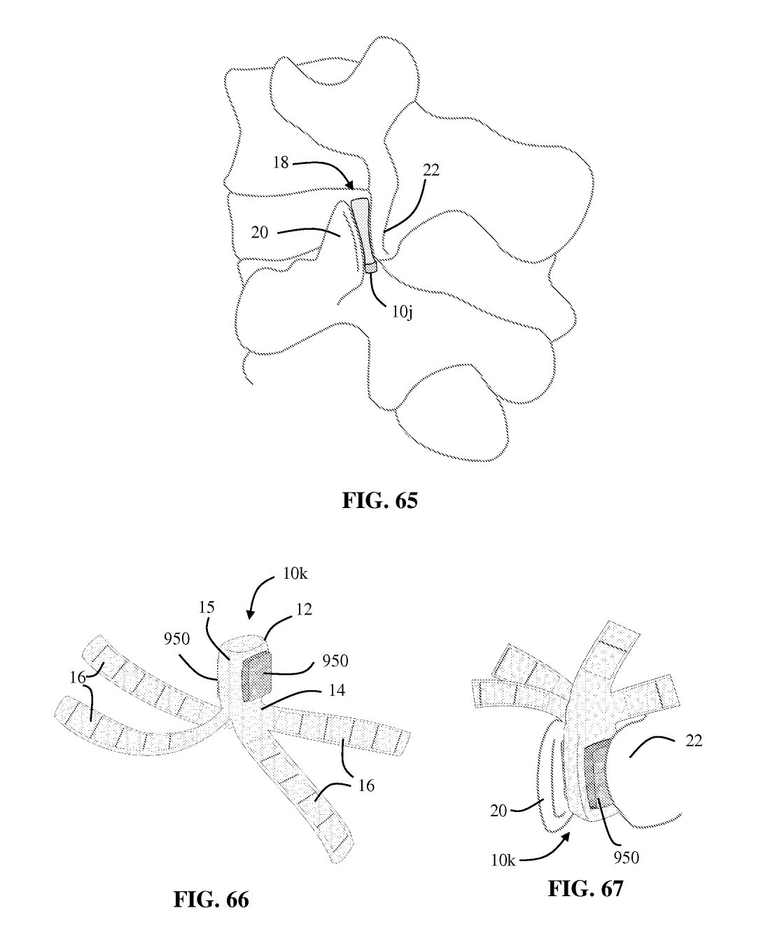

In use, tie cords 900 function not only to secure the facet implant 10j within the facet joint 18, but also to deliver the implant 10j to the facet joint. To accomplish this, a bore 916 is first formed through the facet surface 21 of the superior articular process 20 of the inferior vertebra. The tie cord is threaded through aperture 910 of screw 902, and the screw 902 is then threadedly inserted into the bore 916. The screw 902 is dimensioned such that the shaped engagement element 908 remains outside the bore 916 when the screw 902 has been fully seated. Once screw 902 has been seated within the superior articular process 20, the tie cords 900 are passed through apertures 912 of implant 10j. The implant 10j is then advanced along the tie cords 900 into the facet joint 18. When the implant 10j has been fully inserted within the facet joint 18, the shaped engagement element 908 of the screw 902 is nestled within the recess 914 of the spacer 12. Once the implant 10j has been preferentially seated within the facet joint 18, the tie cords 900 may be tied to secure the implant 10j in place, and excess tie cord 900 may then be severed and removed. FIG. 65 illustrates the implant 10j after implantation within the facet joint 18.

FIGS. 66-70 illustrate an example of a facet implant 10k according to a twelfth embodiment of the present invention. Implant 10k is similar to implant 10 of FIG. 1, and includes a spacer 12 and encapsulating jacket 14. In the example shown in FIG. 66, the jacket 14 includes a body portion 15 that at least partially surrounds the spacer 12. The attachment flanges 16 extend from one end of the body portion 15 such that upon insertion within a facet joint, the flanges 16 will all extend outside the joint in a similar manner. The body portion 15 includes an additional pad 950 that includes a fusion-inducing biologic agent, such as bone morphogenic protein (BMP), stem cell based material, calcium hydroxyapatite, demineralized bone matrix, or Formagraft® offered by NuVasive. Pad 950 including the biologic agent may be provided on either side or both sides of body portion 15.

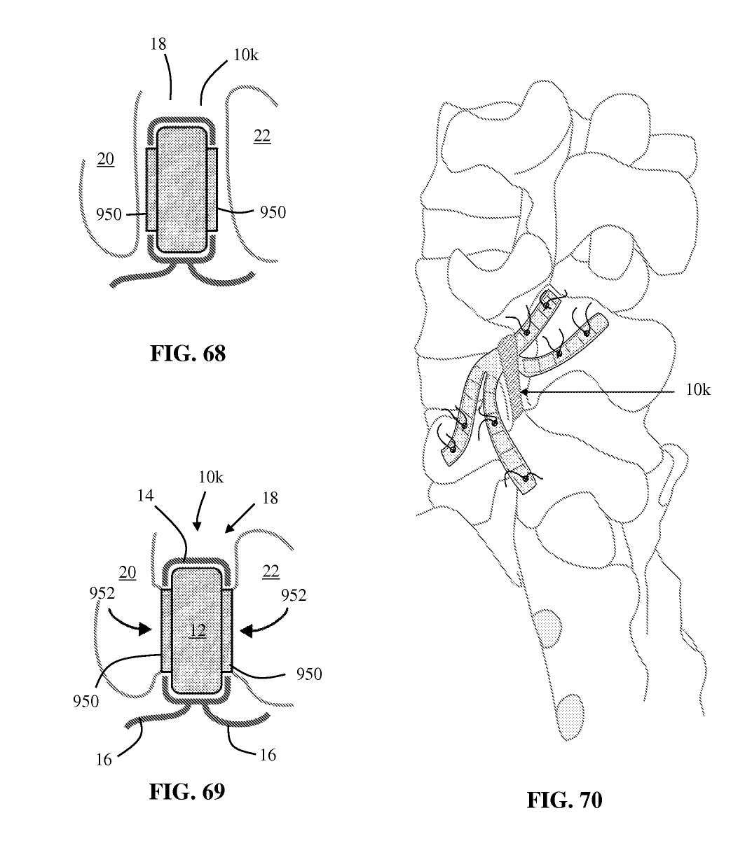

As shown in FIGS. 67-68, the implant 10k is inserted into the facet joint 18 such that the pads 950 are in contact with articular processes 20, 22 forming the facet joint 18. Providing the pad 950 on both sides, as shown by example in FIGS. 66-70, encourages fusion of the implant with the facet joint. The degree of fusion that occurs may be controlled depending on the needs of the user, as described in relation to several of the examples presented above. As shown in FIG. 69, fusion may be achieved at least with the encapsulating jacket 14 such that any facet motion that occurs is within the implant 10k.

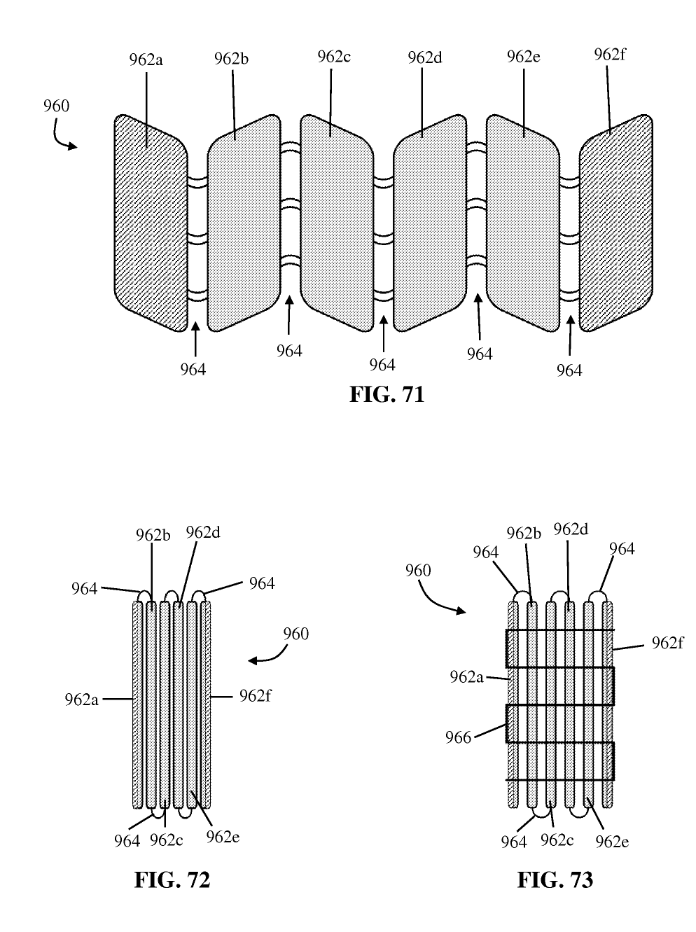

FIGS. 71-77 illustrate an example of a spacer 960 that provides for internal movement within a facet implant such as any of the examples discussed above. The spacer 960 may be provided with or without an encapsulating jacket. The spacer 960 is similar to those shown and described in the above-referenced '944 PCT Application. The spacer 960 is comprised of a plurality of textile layers, for example six layers 962a-962f coupled by a plurality of hinge regions 964. Spacer 960 is provided by example as assembling in an accordion-like manner, however other assemblies are possible. For example, the spacer 960 may be formed from a plurality of individual textile layers consecutively stacked upon one another and/or a single continuous textile sheet folded upon itself to form a plurality of stacked textile layer regions. As shown in FIG. 72, upon assembling the spacer 960 will comprise a pair of “outside” textile layers 962a, 962f separated by a number of “interior” textile layers 962b-962e. As shown in FIG. 73, a supplemental stitching 966 may provided through the various textile layers 962a-962f to tether the layers together and increase stability of the implant.

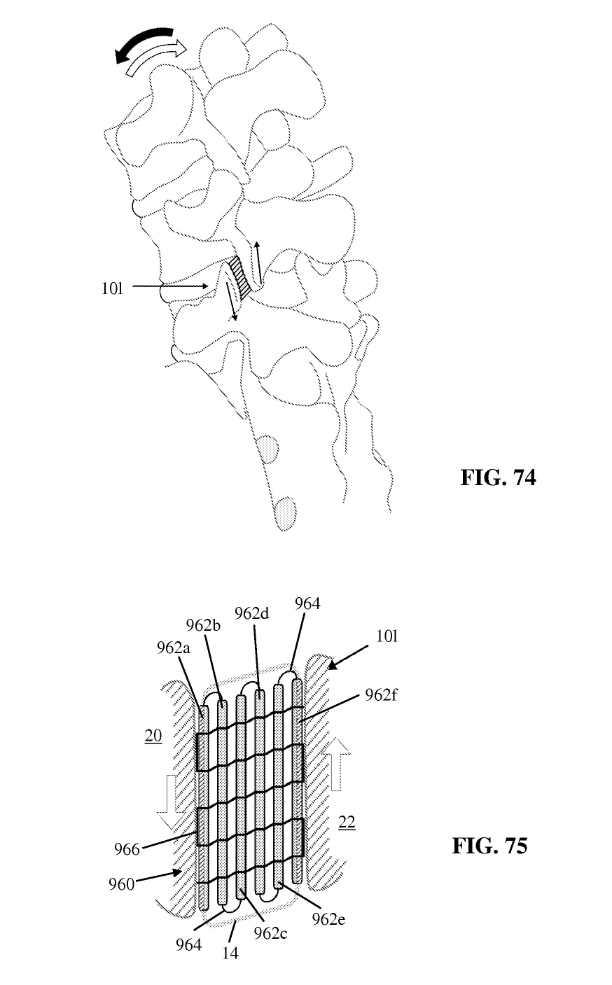

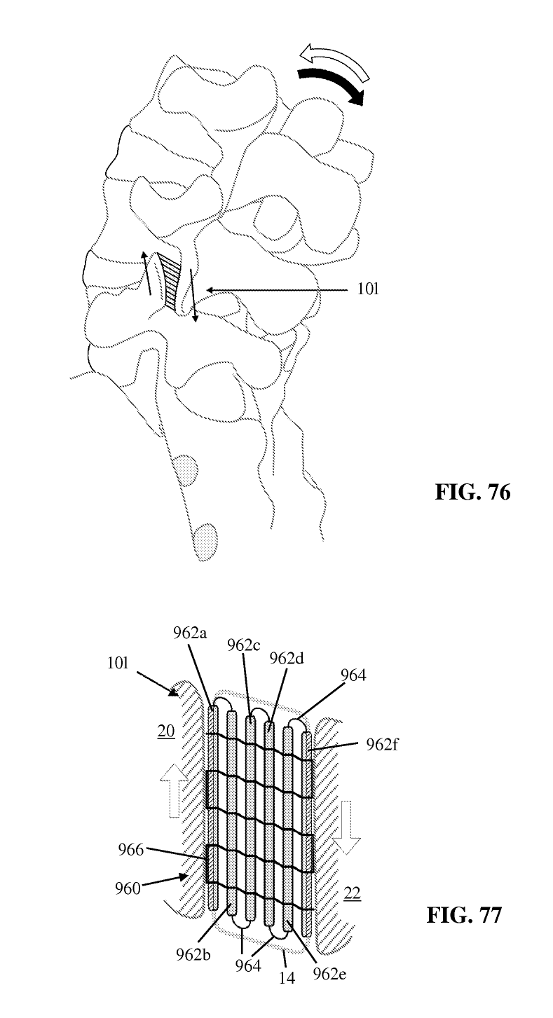

Textile layers 962a-962f may be provided in any number and configuration without departing from the scope of the present invention. In the present example, interior textile layers 962b-962e may be untreated or in the alternative treated with an anti-fusion agent in order to prevent any tissue and/or bony ingrowth through those layers. Furthermore, the layers 962b-962e may be chemically treated or manufactured such that they are capable of moving relative to one another. The outside textile layers 962a, 962f are formed from or treated with fusion-inducing materials to cause tissue and/or bony ingrowth between the bone and the specific outside textile layers 962a, 962f. The result is a facet implant 101 including a layered spacer 960 that achieves a textile-bone fusion interface with the facet surface of the superior articular process 20 of a first vertebra and a textile-bone fusion interface with the facet surface of the inferior articular process 22 of a second vertebra. However, facet motion is retained due to the capability of the interior layers 962b-962e to move or slide relative to one another in response to movement of the articular processes 20, 22. For example, FIGS. 74-75 show the motion of the spine (FIG. 74) and corresponding movement of the spacer 960 (FIG. 75) during spinal flexion. FIGS. 76-77 show the motion of the spine (FIG. 76) and corresponding movement of the spacer 960 (FIG. 77) during spinal extension. In either case, the spacer 960 allows for a “controlled slippage” of the interior textile layers 962b-962e such that at least partial motion within the facet joint may be preserved. Movement of the layers 962b-962e is controlled due to the hinge regions 964 and supplemental stitching 966 as well as an encapsulating jacket 14 (if provided), all of which function to limit the range of motion of the textile layer regions 962b-962e.

Many of the facet implant examples described above encourage at least some tissue and/or bony ingrowth in order to either secure the implant in place or promote complete fusion of the facet joint. Upon successful tissue and/or bony ingrowth, biodegradation, bioresorbtion, bioabsorbtion, bioabsorption, and/or bioerosion of the implant or portions thereof may be encouraged depending upon the desired motion preservation characteristics of the facet joint. For the purposes of this disclosure, bioresorbtion is meant to include any biological process (including those delineated above) in which at least a portion of the fabric component of the implant disappears or becomes detached from the rest of the implant.

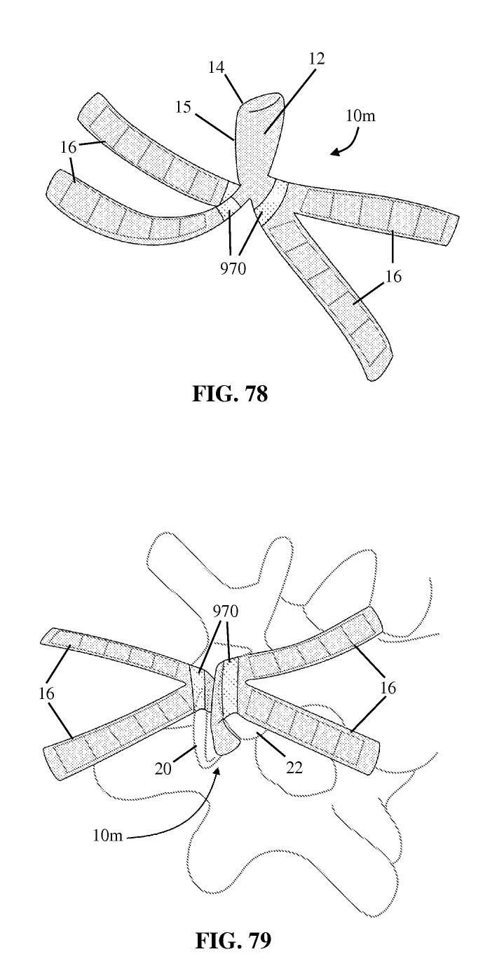

FIGS. 78-81 illustrate an example of a facet implant 10m according to a fourteenth embodiment of the present invention. Implant 10m is similar to implant 10 of FIG. 1, and includes a spacer 12 and encapsulating jacket 14. In the example shown in FIG. 78, the jacket 14 includes a body portion 15 that at least partially surrounds the spacer 12. The attachment flanges 16 extend from one end of the body portion 15 such that upon insertion within a facet joint, the flanges 16 will all extend outside the joint in a similar manner. The encapsulating fabric 14 of the implant 10m includes a portion (e.g. a strip) of bioresorbable fabric 970 on each flange 16 adjacent to the body portion 15. As such, over time the bioresorbable fabric 970 will disappear, causing the body portion 15 and flanges 16 to become detached from one another. The flanges 16 may be secured to the relevant bone portions using any suitable means of attachment, for example including but not limited to bone screws, staples, sutures, nails, buttons, anchors, and/or adhesives.

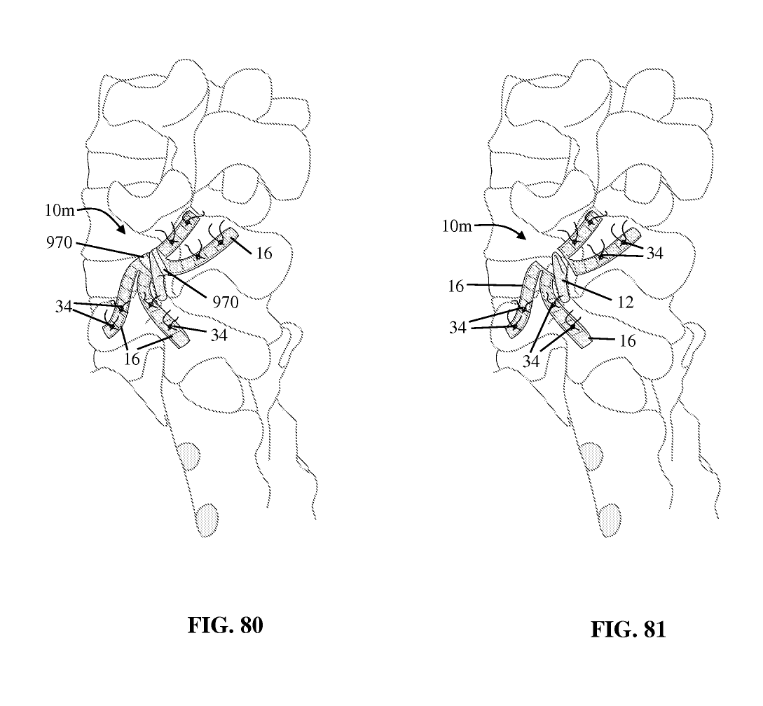

FIG. 79 illustrates the implant 10m including bioresorbable portions 970 inserted between a superior articular process 20 and inferior articular process 22 of adjacent vertebrae before the flanges 16 have been attached to the bone. FIG. 80 illustrates the implant 10m after the flanges 16 have been secured to bone with sutures 34. FIG. 81 illustrates the implant 10m in position after resorbtion of the bioresorbable fabric portions 970 has occurred. The spacer 12 is thus detached from the flanges 16 and left within the facet joint.

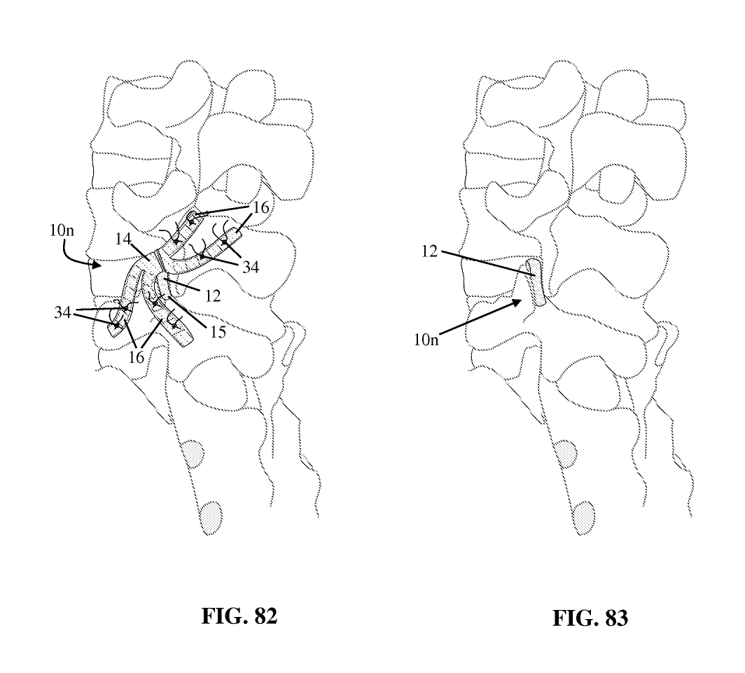

FIGS. 82-83 illustrate an example of a facet implant 10n according to a fifteenth embodiment of the present invention. Implant 10n is similar to implant 10 of FIG. 1, and includes a spacer 12 and encapsulating jacket 14. In the example shown in FIG. 82, the jacket 14 includes a body portion 15 that at least partially surrounds the spacer 12. The attachment flanges 16 extend from one end of the body portion 15 such that upon insertion within a facet joint, the flanges 16 will all extend outside the joint in a similar manner. In this example, the portions of the encapsulating fabric 14 forming the flanges 16 are entirely bioresorbable, and after resorbtion only the spacer 12 is left within the facet joint (FIG. 83).

Regarding the methods of using all examples of facet implants disclosed herein, it will be understood that several method steps are inherent to performing surgery, and thus have been omitted from each description of use above. However, these steps may be integral in the use of the devices disclosed herein, including but not limited to creating an incision in a patient's skin, distracting and retracting tissue to establish an operative corridor to the surgical target site, advancing the implant through the operative corridor to the surgical target site, removing instrumentation from the operative corridor upon insertion of the implant into the target facet joint, and closing the surgical wound.

Although described with respect to specific examples of the different embodiments, any features of the facet implants disclosed herein by way of example only may be applied to any of the embodiments without departing from the scope of the present invention. Furthermore, procedures described for example only involving specific structure (e.g. superior articular process) may be applied to another structure (e.g. inferior articular process) without departing from the scope of the present invention.

While this invention has been described in terms of a best mode for achieving this invention's objectives, it will be appreciated by those skilled in the art that variations may be accomplished in view of these teachings without deviating from the spirit or scope of the invention.

1. A system for repairing a facet joint, said facet joint existing between a superior articular process of a first vertebra and an inferior articular process of a second vertebra, comprising:
a biocompatible implant configured for positioning within said facet joint, said implant including a spacer and an attachment element; and
a fixation element configured to engage said attachment element to secure said implant to at least one of said superior and inferior articular processes.

2. The system of claim 1, wherein said implant further includes a textile jacket configured to at least partially encapsulate said spacer.

3. The system of claim 2, wherein said textile jacket is formed from at least one fibrous material from the group consisting of polyester fiber, polypropylene, polyethylene, ultra high molecular weight polyethylene, poly-ether-ether-ketone, carbon fiber, glass, glass fiber, polyaramide, metal, copolymers, polyglycolic acid, polylactic acid, biodegradable fibers, nylon, silk, cellulosic and polycaprolactone fibers.

4. The system of claim 2, wherein said textile jacket comprises a structure formed by at least one of embroidery, weaving, three-dimensional weaving, knitting, three-dimensional knitting, injection molding, compression molding, cutting woven fabrics and cutting knitted fabrics.

5. The system of claim 2, wherein said attachment element comprises at least one flange extending from said textile jacket.

6. The system of claim 5, wherein said at least one flange is at least one of attached to and integral with said textile jacket.

7-8. (canceled)

9. The system of claim 2, wherein said textile jacket further includes a pad positioned upon an engagement interface between said textile jacket and one of said superior and inferior articular process, said pad including a biologic agent to encourage fusion between said textile jacket and said articular process.

10. (canceled)

11. The system of claim 2, wherein the textile jacket includes at least one pocket dimensioned to receive a portion of an insertion tool.

12. (canceled)

13. The system of claim 1, wherein said spacer comprises a textile fabric material formed from at least one of synthetic fibers and natural fibers.

14-19. (canceled)

20. The system of claim 0, wherein said spacer comprises a stack of textile layer regions stacked on top of one another.

21. The system of claim 20, wherein said stack is formed from at least one of a plurality of individual textile layer elements consecutively stacked on top of one another, a plurality of hingedly connected individual textile layer regions folded on top of one another, and a single continuous textile sheet folded upon itself to form a plurality of stacked textile layer regions.

22-26. (canceled)

27. The system of claim 1, wherein said attachment element comprises at least one of a bone-engaging surface of said spacer, a protrusion extending generally orthogonally from said spacer, and an aperture extending through said spacer, said aperture configured to receive said fixation element therethrough.

28. The system of claim 27, wherein said protrusion comprises at least one of a flexible strip, at least one cord, and a rigid post.

29-63. (canceled)

64. A method of repairing a facet joint, said facet joint existing between a superior articular process of a first vertebra and an inferior articular process of a second vertebra, each of said superior and inferior articular processes having an interior facet surface forming a portion of the facet joint and an exterior surface opposite said interior facet surface, comprising the steps of:
(a) creating an operative corridor to access said facet joint;
(b) forming an aperture in at least one of said first and second articular processes, said aperture extending from said interior facet surface to said exterior surface of said articular process;
(c) advancing a biocompatible implant through said operative corridor toward said facet joint, said implant comprising a spacer including at least one attachment element extending therefrom, said attachment element configured to engage a fixation element to secure said implant within said facet joint;
(d) positioning said implant within said facet joint such that said attachment element traverses said aperture from said interior facet surface and at least a portion of said attachment element protrudes from said aperture on said exterior surface of said articular process;
(e) securing said implant within said facet joint by engaging a fixation element with said attachment element portion protruding from said aperture; and
(f) closing said operative corridor.

65. The method of claim 64, wherein said implant further includes a textile jacket configured to at least partially encapsulate said spacer.

66-67. (canceled)

68. The method of claim 65, wherein said attachment element comprises at least one flange extending from said textile jacket.

69. The method of claim 68, wherein said at least one flange is at least one of attached to and integral with said textile jacket.

70-71. (canceled)

72. The method of claim 65, wherein said textile jacket further includes a pad positioned upon an engagement interface between said textile jacket and one of said superior and inferior articular process, said pad including a biologic agent to encourage fusion between said textile jacket and said articular process.

73. (canceled)

74. The method of claim 65, wherein the textile jacket includes at least one pocket dimensioned to receive a portion of an insertion tool.

75-93. (canceled)

94. A method of repairing a facet joint, said facet joint existing between a superior articular process of a first vertebra and an inferior articular process of a second vertebra, each of said superior and inferior articular processes having an interior facet surface forming a portion of the facet joint and an exterior surface opposite said interior facet surface, comprising the steps of:
(a) creating an operative corridor to access said facet joint;
(b) forming an aperture at least partially through one of said first and second articular processes;
(c) inserting a fixation element into said aperture, said fixation element having at least one tie-cord extending therefrom;
(d) engaging said at least one tie-cord with a biocompatible implant, said implant comprising a spacer including a recess formed therein, said recess configured to receive at least a portion of said fixation element to secure said implant within said facet joint;
(e) advancing said biocompatible implant through said operative corridor toward said facet joint;
(f) securing said implant within said facet joint by tying said tie-cords around at least a portion of said implant; and
(g) closing said operative corridor.

95-123. (canceled)
